(19) United States
(12) Patent Application Publication
(10) Pub. No.: US 2008/0146612 A1
(43) Pub. Date: Jun. 19, 2008

(54) Novel Biaromatic Compounds, Inhibitors of the P2X7-Receptor

(75) Inventors: Toby Thompson, Leicestershire (GB); Paul Willis, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/814,530

(22) PCT Filed: Jan. 25, 2006
(86) PCT No.: PCT/SE2006/000108

§ 371 Date: Jul. 23, 2007

(30) Foreign Application Priority Data
Jan. 27, 2005 (SE) 0500218-3
Apr. 8, 2005 (SE) 0500793-5

(51) Int. Cl. A61K 31/4545; C07C 63/33; C07D 211/86; C07D 211/04; A61P 19/02; A61P 11/06; A61P 9/10; A61K 31/195; A61K 31/455

(52) U.S. Cl. 514/318; 562/492; 546/326; 546/194; 514/567; 514/354

(57) ABSTRACT

The invention provides compounds of formula (I), or a pharmaceutically acceptable salt thereof, wherein Ar¹ represents a group (II), (III), (IV) or (V), and A, Ar², n, R¹, R², R³, R⁴ and R⁵ are as defined in the specification; a process for their preparation; pharmaceutical compositions containing them; and their use in therapy.

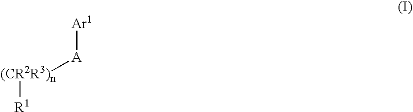

The present invention relates to new biaromatic derivatives, a process for their preparation, pharmaceutical compositions containing them, a process for preparing pharmaceutical compositions and their use in therapy.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

Compounds effective as P2X₇ receptor antagonists are of interest for use in the treatment of inflammatory, immune or cardiovascular diseases, in the aetiologies of which the P2X₇ receptor may play a role. Accordingly, there is a need for P2X₇ receptor antagonists having improved pharmaceutical properties.

The present invention provides a new class of P2X₇ antagonist which comprises a substituted biaromatic group. These novel compounds display excellent properties for use as P2X₇ receptor antagonists in the treatment of inflammatory, immune or cardiovascular diseases. Whilst P2X₇ antagonists have been described previously, for example in WO 00/61569, WO 01/42194, WO 01/44170, WO 01/44213, WO 01/46200, WO 01/94338, WO 03/041707, WO 03/042190, WO 03/042191, WO 03/080579, WO 04/058270, WO 04/058731, WO 04/074224 and WO 04/099146, prior to the present invention there had been no suggestion that compounds comprising the substituted biaromatic group of the present invention would make effective P2X₇ antagonists.

US patent application 2004/0214888 describes carboxylic acid derivatives useful as insulin sensitizers, whilst US patent application 2003/0134885 describes carboxyl-substituted biphenyl ligand activators of PPARgamma receptors. Neither document makes any mention of the P2X₇ receptor.

In accordance with the present invention, there is provided a compound of general formula (I), or a pharmaceutically acceptable salt thereof,

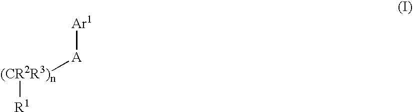

wherein Ar¹ represents a group

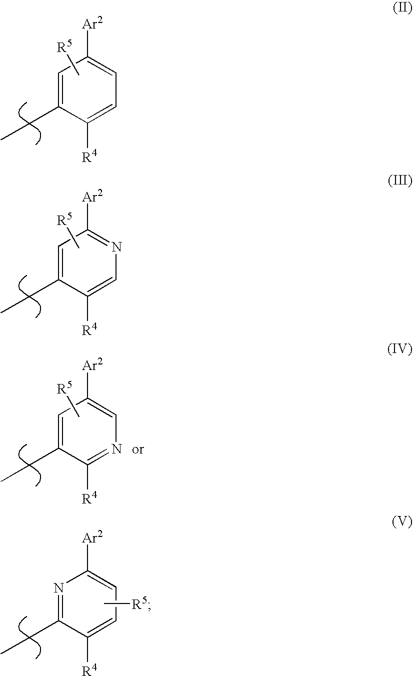

A represents C(O)NH or NHC(O);
R¹ represents a 3- to 9-membered carbocyclic or 4- to 10-membered heterocyclic ring, which carbocyclic ring or heterocyclic ring can be optionally substituted by at least one substituent independently selected from halogen, hydroxyl, cyano, nitro, NR⁶R⁷, C₁₋₆ alkylsulphonyl, C₁₋₆ alkoxy and a C₁₋₆ alkyl group which C₁₋₆ alkyl group can be optionally substituted by at least one substituent independently selected from halogen and hydroxyl;
n is 0, 1, 2 or 3;
within each grouping, CR²R³, R² and R³ each independently represent hydrogen, halogen, phenyl or a C₁₋₆ alkyl group, or R² and R³ together with the carbon atom to which they are both attached form a 3- to 8-membered cycloalkyl ring;
one of R⁴ and R⁵ represents halogen, nitro, NR⁶R⁷, hydroxyl, C₁₋₆ alkoxy optionally substituted by at least one halogen, or a C₁₋₆ alkyl group optionally substituted by at least one halogen, and the other of R⁴ and R⁵ represents hydrogen, halogen or a C₁₋₆ alkyl group optionally substituted by at least one halogen;
Ar² represents phenyl substituted by at least one substituent independently selected from carboxyl, MC₁₋₆ alkylCO₂H, C₁₋₆ alkylsulphonylaminocarbonyl, C(O)NHOH, NHR⁸, R⁹, XR¹⁰ and NR¹⁷R¹⁸, or Ar² represents a 5- or 6-membered heteroaromatic ring comprising from 1 to 2 heteroatomns independently selected from nitrogen, oxygen and sulphur, which heteroaromatic ring is substituted by at least one substituent independently selected from carboxyl, MC₁₋₆ alkylCO₂H, C₁₋₆ alkylsulphonylaminocarbonyl, C(O)NHOH, NHR⁸ and NR¹⁹R²⁰;
wherein the phenyl or heteroaromatic ring Ar² can further be optionally substituted by at least one substituent independently selected from halogen, nitro, NR⁶R⁷, S(O)₀₋₂R¹¹, C₁₋₆ alkoxy optionally substituted by at least one halogen, and a C₁₋₆ alkyl group which C₁₋₆ alkyl group can be optionally substituted by at least one substituent independently selected from halogen, hydroxyl, NR⁶R⁷, SO₂NR⁶R⁷, NR¹¹SO₂R¹¹, NHCOR¹¹ and CONR⁶R⁷;
R⁸ represents CN, C₁₋₆ alkoxycarbonyl, C₁₋₆ alkylaminosulphonyl, or (di)-C₁₋₆ alkylaminosulphonyl;
R⁹ and R¹⁰ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen and sulphur, which heterocyclic ring is substituted by at least one substituent independently selected from hydroxyl, ═O and ═S, and which heterocyclic ring may further be optionally substituted by at least one substituent independently selected from halogen, nitro, amino, cyano, C₁₋₆ alkylsulphonyl, C₁₋₆alkoxycarbonyl and a C₁₋₆ alkyl group which C₁₋₆ alkyl group can be optionally substituted by at least one substituent independently selected from halogen, hydroxyl and amino;
M represents a bond, oxygen, S(O)₀₋₂ or NR¹;
X represents oxygen, S(O)₀₋₂, NR¹¹, C₁₋₆ alkylene, O(CH₂)₁₋₆, NR¹¹(CH₂)₁₋₆ or S(O)₀₋₂(CH₂)₁₋₆;
R⁶ and R⁷ each independently represent a hydrogen atom or a C₁₋₆ alkyl group optionally substituted by at least one substituent independently selected from hydroxyl, halogen and C₁₋₆ alkoxy, or R⁶ and R⁷ together with the nitrogen atom to which they are attached form a 3- to 8-membered saturated heterocyclic ring;
R¹¹ represents a hydrogen atom or a C₁₋₆ alkyl group optionally substituted by at least one substituent independently selected from hydroxyl, halogen and C₁₋₆ alkoxy;
R¹⁷ and R¹⁸ together with the nitrogen atom to which they are attached form a 3- to 8-membered saturated heterocyclic ring, which heterocyclic ring is substituted with at least one substituent independently selected from carboxyl, MC₁₋₆ alkylCO₂H, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁₋₆ alkoxy optionally substituted by at least one halogen, and a C₁₋₆ alkyl group which C₁₋₆ alkyl group can be optionally substituted by at least one substituent independently selected from halogen and hydroxyl;
R¹⁹ and R²⁰ together with the nitrogen atom to which they are attached form a 3- to 8-membered saturated heterocyclic ring, which heterocyclic ring is substituted with at least one substituent independently selected from carboxyl, MC₁₋₆ alkylCO₂H, C₁₋₆ alkylsulphonylaminocarbonyl, C(O)NHOH and NHR⁸, and which 3- to 8-membered saturated heterocyclic ring can further be optionally substituted by at least one substituent independently selected from hydroxyl, halogen, C₁₋₆ alkoxy optionally substituted by at least one halogen, and a C₁₋₆ alkyl group which C₁₋₆ alkyl group can be optionally substituted by at least one substituent independently selected from halogen and hydroxyl;
provided that the compound of formula (I) is not
(3-{4-chloro-3-[(1-hydroxy-cycloheptylmethyl)-carbamoyl]-phenyl}-5-methyl-pyrazol-1-yl)-acetic acid,
3-[4-methoxy-3-({[4-(trifluoromethyl)benzyl]amino}carbonyl)phenyl]benzoic acid, or
3-[4-methoxy-3-({[2,4-dichlorobenzyl]amino}carbonyl)phenyl]benzoic acid.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

It will be understood that certain compounds of the present invention may exist in solvated, for example hydrated, as well as unsolvated forms. It is to be understood that the present invention encompasses all such solvated forms.

In the context of the present specification, a ‘Carbocyclic’ ring is an unsaturated, saturated or partially saturated mono- or bicyclic ring, containing only carbon ring atoms, and may have aliphatic or aromatic properties. A ‘Heterocyclic’ ring is an unsaturated, saturated or partially saturated mono- or bicyclic ring, at least one atom of which is a heteroatom selected from oxygen, sulphur or nitrogen, and may have aliphatic or aromatic properties. ‘Heteroaromatic’ denotes aromatic rings, at least one atom of which is a heteroatom selected from oxygen, sulphur or nitrogen. ‘Cycloalkyl’ denotes saturated alkyl rings. Unless otherwise indicated an alkyl group may be linear or branched. Where a group is described as being ‘optionally substituted by at least one substituent’, the group may be unsubstituted or carry one or more (e.g. one, two or three) substituents.

In an embodiment of the invention, A represents NHC(O). In another embodiment of the invention, A represents C(O)NH.

R¹ represents a 3- to 9-membered carbocyclic or 4- to 10-membered heterocyclic ring, which carbocyclic or heterocyclic ring can be optionally substituted by af least one substituent independently selected from halogen (e.g. fluorine, chlorine, bromine or iodine), hydroxyl, cyano, nitro, NR⁶R⁷, C₁₋₆ alkylsulphonyl (e.g. MeSO₂—), C₁₋₆, preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl.

In an embodiment of the invention R¹ represents a 3- to 9-membered aliphatic carbocyclic ring optionally substituted by at least one substituent (e.g. one, two or three) independently selected from halogen (e.g. fluorine, chlorine, bromine or iodine), hydroxyl, cyano, nitro, NR⁶R⁷, C₁₋₆ alkylsulphonyl (e.g. MeSO₂—), C₁₋₆, preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl. In a further aspect of this embodiment R¹ represents cyclopropyl, cyclobutyl, cyclopentyl, cyclohexyl, cycloheptyl, cycloctyl or bicycloheptyl, each of which can be optionally substituted by at least one substituent (e.g. one, two or three) independently selected from halogen (e.g. fluorine, chlorine, bromine or iodine), hydroxyl, C₁₋₆, preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl. Examples of groups according to this embodiment include cyclopropyl, cyclobutyl, cyclopentyl, cyclohexyl, cycloheptyl, cycloctyl,

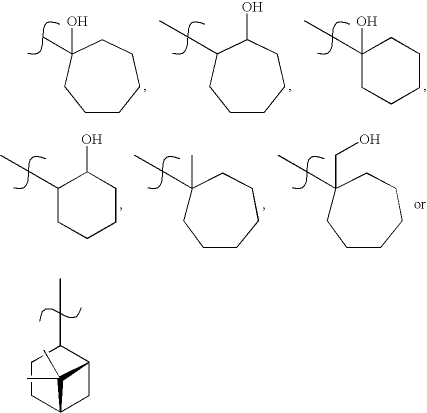

In another embodiment of the invention, R¹ represents phenyl optionally substituted by at least one substituent (e.g. one, two or three) independently selected from halogen (e.g. fluorine, chlorine, bromine or iodine), hydroxyl, cyano, nitro, NR⁶R⁷, C₁₋₆ alkylsulphonyl (e.g. MeSO₂—), C₁₋₆, preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl. Examples of groups R¹ according to this embodiment are phenyl or 2-chlorophenyl.

In another embodiment of the invention R¹ represents a 4- to 10-membered heteroaromatic ring containing from 1 to 3, or 1 to 2 heteroatoms, selected from nitrogen, oxygen and sulphur, which heteroaromatic ring can be optionally substituted with at least one substituent (e.g. one, two or three) independently selected from halogen (e.g. fluorine, chlorine, bromine or iodine), hydroxyl, cyano, nitro, NR⁶R⁷, C₁₋₆ alkylsulphonyl (e.g. MeSO₂—), C₁₋₆ preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl. Examples of heteroaromatic rings according to this embodiment include pyridinyl, pyridazinyl, pyrazinyl, pyrimidinyl, pyrazolyl and quinolinyl.

In another embodiment of the invention R¹ represents a monocyclic aliphatic 5- to 8-membered heterocyclic ring containing 1 to 3, or 1 to 2 heteroatoms selected from nitrogen, oxygen and sulphur, which heterocyclic ring may be optionally substituted with at least one substituent (e.g. one, two or three) independently selected from halogen (e.g. fluorine, chlorine, bromine or iodine), hydroxyl, cyano, nitro, NR⁶R⁷, C₁₋₆ alkylsulphonyl (e.g. MeSO₂—), C₁₋₆, preferably C₁₋₄, alkoxy (e.g. methoxy, ethoxy, n-propoxy, n-butoxy, n-pentoxy or n-hexoxy) and a C₁₋₆, preferably C₁₋₄, alkyl group (e.g. methyl, ethyl, n-propyl, isopropyl, n-butyl, isobutyl, tert-butyl, n-pentyl or n-hexyl) which C₁₋₆ alkyl group can be optionally substituted by at least one substituent (e.g. one, two or three) independently selected from halogen (e.g. fluorine, chlorine, bromine or iodine) and hydroxyl. Examples of heterocyclic rings according to this embodiment include pyrrolidinyl, piperidinyl, piperazinyl, homopiperazinyl and homopiperidinyl.

In an embodiment of the invention, n is 0, 1 or 2. In another embodiment of the invention n is 0. In a further embodiment of the invention n is 1 or 2.

Within each grouping, CR²R³, R² and R³ each independently represent hydrogen, halogen (e.g. fluorine, chlorine, bromine or iodine), phenyl or a C₁₋₆, preferably C₁₋₄, alkyl group (e.g. methyl, ethyl, n-propyl, isopropyl, n-butyl, isobutyl, tert-butyl, n-pentyl or n-hexyl), or R² and R³ together with the carbon atom to which they are both attached form a 3- to 8-membered cycloalkyl ring (e.g. cyclopropyl, cyclobutyl, cyclopentyl or cyclohexyl).

In an embodiment of the invention R² and R³ each independently represent hydrogen, C₁₋₄ alkyl, or R² and R³ together with the carbon atom to which they are both attached form a cyclopropyl ring. In another embodiment of the invention, R² and R³ each independently represent hydrogen.

One of R⁴ and R⁵ represents halogen (e.g. fluorine, chlorine, bromine or iodine), nitro, NR⁶R⁷, hydroxyl, C₁₋₆, preferably C₁₋₄, alkoxy (e.g. methoxy, ethoxy, n-propoxy, n-butoxy, n-pentoxy or n-hexoxy) optionally substituted by at least one (e.g. one, two or three) halogen (e.g. fluorine, chlorine, bromine or iodine) or a C₁₋₆, preferably C₁₋₄, alkyl group (e.g. methyl, ethyl, n-propyl, isopropyl, n-butyl, isobutyl, tert-butyl, n-pentyl or n-hexyl), optionally substituted by at least one (e.g. one, two or three) halogen (e.g. fluorine, chlorine, bromine or iodine), and the other of R⁴ and R⁵ represents hydrogen, halogen (e.g. fluorine, chlorine, bromine or iodine) or a C₁₋₆, preferably C₁₋₄, alkyl group (e.g. methyl, ethyl, n-propyl, isopropyl, n-butyl, isobutyl, tert-butyl, n-pentyl or n-hexyl) optionally substituted by at least one (e.g. one, two or three) halogen (e.g. fluorine, chlorine, bromine or iodine).

In an embodiment of the invention, R⁴ represents halogen, nitro, NH₂, hydroxyl, or a C₁₋₄ alkyl optionally substituted by one to three halogen substituents; and R⁵ represents a hydrogen atom.

In an embodiment of the invention, Ar¹ represents a group (II) or (III).

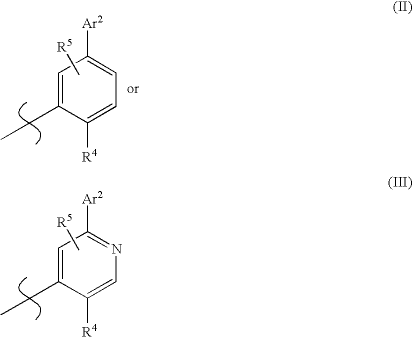

In an embodiment of the invention, Ar¹ represents a group (II)

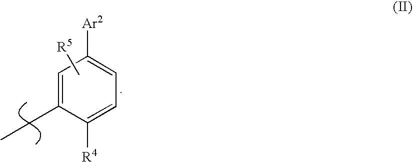

According to the present invention, Ar² represents phenyl substituted by at least one (e.g. one or two) substituent independently selected from carboxyl, MC₁₋₆ alkylCO₂H, C₁₋₆ alkylsulphonylaminocarbonyl (e.g. MeSO₂NHCO—), C(O)NHOH, NHR⁵, R⁹, XR¹⁰ and NR¹⁷R¹⁸, or Ar² represents a 5- or 6-membered heteroaromatic ring comprising from 1 to 2 heteroatoms independently selected from nitrogen, oxygen and sulphur, which heteroaromatic ring is substituted by at least one (e.g. one or two) substituent independently selected from carboxyl, MC₁₋₆ alkylCO₂H, C₁₋₆ alkylsulphonylaminocarbonyl (e.g. MeSO₂NHCO—), C(O)NHOH, NHR⁵ and NR¹⁹R²⁰;

wherein the phenyl or heteroaromatic ring Ar² can further be optionally substituted by at least one substituent (e.g. one or two) independently selected from halogen (e.g. fluorine, chlorine, bromine or iodine), nitro, NR⁶R⁷, S(O)₀₋₂R¹¹, C₁₋₆ preferably C₁₋₄, alkoxy (e.g. methoxy, ethoxy, n-propoxy, n-butoxy, n-pentoxy or n-hexoxy) which alkoxy group can be optionally substituted by at least one (e.g. one, two or three) halogen (e.g. fluorine, chlorine, bromine or iodine), and a C₁₋₆, preferably C₁₋₄, alkyl group (e.g. methyl, ethyl, n-propyl, isopropyl, n-butyl, isobutyl, tert-butyl, n-pentyl or n-hexyl) which alkyl group can be optionally substituted by at least one (e.g. one, two or three) substituent independently selected from halogen (e.g. fluorine, chlorine, bromine or iodine), hydroxyl, NR⁶R⁷, SO₂ NR⁶R⁷, NR¹¹SO₂R¹¹, NHCOR¹¹ and CONR⁶R⁷.

In an embodiment of the invention, Ar² represents phenyl, optionally substituted as defined herein above.

In another embodiment of the invention Ar² represents a 5- to 6-membered heteroaromatic ring selected from pyrryl, thienyl, furanyl, imidazolyl, thiazolyl, oxazolyl, isothiazolyl, isoxazolyl, pyrazolyl, pyridyl, pyridazinyl, pyrimidinyl and pyrazinyl, which heteroaromatic ring is optionally substituted as defined herein above.

In a further embodiment of the invention, Ar² represents pyridyl, optionally substituted as defined herein above.

In an embodiment of the invention, Ar² is substituted by a substituent selected from carboxyl, MC₁₋₆ alkylCO₂H and C₁₋₆ alkylsulphonylaminocarbonyl.

In another embodiment of the invention, Ar² is substituted by carboxyl.

In another embodiment of the invention Ar² is phenyl substituted by a substituent NR¹⁷R¹⁸ wherein R¹⁷ and R¹⁸ together with the nitrogen atom to which they are attached form a 3- to 8-membered saturated heterocyclic ring which heterocyclic ring is substituted with at least one substituent independently selected from carboxyl, MC₁₋₆ alkylCO₂H, and C₁₋₆ alkylsulphonylaminocarbonyl. In a further aspect of this embodiment the heterocyclic ring of NR¹⁷R¹⁵ is substituted by carboxyl.

In another embodiment of the invention Ar² is pyridyl substituted by a substituent NR¹⁹R²⁰ wherein R¹⁹ and R²⁰ together with the nitrogen atom to which they are attached form a 3- to 8-membered saturated heterocyclic ring which heterocyclic ring is substituted with at least one substituent independently selected from carboxyl, MC₁₋₆ alkylCO₂H, and C₁₋₆ alkylsulphonylaminocarbonyl. In a further aspect of this embodiment the heterocyclic ring of NR¹⁹R²⁰ is substituted by carboxyl.

In an embodiment of the invention, M represents a bond or oxygen. In another embodiment of the invention, M represents a bond.

In an embodiment of the invention X represents oxygen or C₁₋₄ alkylene.

R⁸ represents CN, C₁₋₆, preferably C₁₋₄, alkoxycarbonyl (e.g. methoxy-, ethoxy-, n-propoxy-, n-butoxy-, n-pentoxy- or n-hexoxycarbonyl), C₁₋₆ preferably C₁₋₄, alkylaminosulphonyl (e.g. MeNHSO₂ or EtNHSO₂—), or (di)-C₁₋₆, preferably C₁₋₄, alkylaminosulphonyl (e.g. Me₂NSO₂ or Et₂NSO₂— or EtMeNSO₂—).

R⁹ and R¹⁰ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen and sulphur, which heterocyclic ring is substituted by at least one substituent (e.g. one, two or three) independently selected from hydroxyl, ═O and ═S, and which heterocyclic ring may further be optionally substituted by at least one substituent (e.g. one or two) independently selected from halogen (e.g. chlorine, fluorine, bromine or iodine), nitro, amino, cyano, C₁₋₆, preferably C₁₋₄, alkylsulphonyl (e.g. MeSO₂— or EtSO₂—), C₁₋₆, preferably C₁₋₄, alkoxycarbonyl (e.g. methoxy-, ethoxy-, n-propoxy-, n-butoxy-, n-pentoxy- or n-hexoxycarbonyl), and a C₁₋₆, preferably C₁₋₄, alkyl (e.g. methyl, ethyl, n-propyl, isopropyl, n-butyl, isobutyl, tert-butyl, n-pentyl or n-hexyl) group which C₁₋₆-alkyl group can be optionally substituted by at least one substituent (e.g. one, two or three) independently selected from halogen (e.g. chlorine, fluorine, bromine or iodine), hydroxyl and amino.

When R⁹ and R¹⁰ each independently represent a 5- to 6-membered heterocyclic ring, nitrogen atoms in the heterocyclic ring may carry hydroxyl substituents and sulphur atoms in the ring may be in the form of S, SO (i.e. carrying one ═O substituent) or SO₂ (i.e. carrying two ═O substituents).

Where R⁹ or R¹⁰ represents a 5- to 6-membered heterocyclic ring comprising from 1-4 heteroatoms independently selected from nitrogen, oxygen and sulphur, which heterocyclic ring is substituted by at least one substituent selected from hydroxyl, ═O and ═S, examples include:

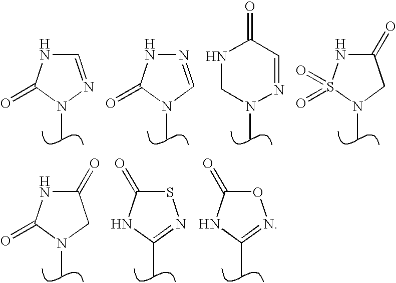

In an embodiment of the invention, R⁹ and R¹⁰ independently represent a 5- to 6-membered heterocyclic ring comprising from 2 to 3 nitrogen atoms and optionally 1 further heteroatom selected from oxygen and sulphur, which heterocyclic ring is substituted by at least one substituent independently selected from hydroxyl, ═O and ═S.

R⁶ and R⁷ each independently represent a hydrogen atom or a C₁₋₆, preferably C₁₋₄, alkyl group (e.g. methyl, ethyl, n-propyl, isopropyl, n-butyl, isobutyl, tert-butyl, n-pentyl or n-hexyl) optionally substituted by at least one (e.g. one, two or three) substituent independently selected from hydroxyl, halogen (e.g. fluorine, chlorine, bromine or iodine) and C₁₋₆ alkoxy, preferably C₁₋₄, alkoxy (e.g. methoxy, ethoxy, n-propoxy, n-butoxy, n-pentoxy or n-hexoxy), or R⁶ and R⁷ together with the nitrogen atom to which they are attached form a 3- to 8-membered saturated heterocyclic ring.

R¹¹ represents a hydrogen atom or a C₁₋₆, preferably C₁₋₄, alkyl group (e.g. methyl, ethyl, n-propyl, isopropyl, n-butyl, isobutyl, tert-butyl, n-pentyl or n-hexyl), optionally substituted by at least one (e.g. one, two or three) substituent independently selected from hydroxyl, halogen (e.g. fluorine, chlorine, bromine or iodine) and C₁₋₆ alkoxy, preferably C₁₋₄, alkoxy (e.g. methoxy, ethoxy, n-propoxy, n-butoxy, n-pentoxy or n-hexoxy).

R¹⁷ and R¹⁸ together with the nitrogen atom to which they are attached form a 3- to 8-membered, or 4- to 7-membered, saturated heterocyclic ring, which heterocyclic ring is substituted with at least one substituent (e.g. one, two or three) independently selected from carboxyl, MC₁₋₆ alkylCO₂H, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁₋₆ alkoxy optionally substituted by at least one halogen, and a C₁₋₆ alkyl group which C₁₋₆ alkyl group can be optionally substituted by at least one substituent independently selected from halogen and hydroxyl. Examples of saturated heterocyclic rings that R¹⁷ and R¹⁸ together with the nitrogen atom to which they are attached may form are rings containing one or two nitrogen atoms, e.g. pyrrolidinyl, piperidinyl, piperazinyl, homopiperazinyl, homopiperidinyl and azetidinyl.

R¹⁹ and R²⁰ together with the nitrogen atom to which they are attached form a 3- to 8-membered, or 4- to 7-membered, saturated heterocyclic ring, which heterocyclic ring is substituted with at least one substituent (e.g. one, two or three) independently selected from carboxyl, MC₁₋₆ alkylCO₂H, C₁₋₆ alkylsulphonylaminocarbonyl, C(O)NHOH and NHR⁸, and which 3- to 8-membered saturated heterocyclic ring can further be optionally substituted by at least one substituent independently selected from hydroxyl, halogen, C₁₋₆ alkoxy optionally substituted by at least one halogen, and a C₁₋₆ alkyl group which C₁₋₆ alkyl group can be optionally substituted by at least one substituent independently selected from halogen and hydroxyl. Examples of saturated heterocyclic rings that R¹⁹ and R²⁰ together with the nitrogen atom to which they are attached may form are rings containing one or two nitrogen atoms, e.g. pyrrolidinyl, piperidinyl, piperazinyl, homopiperazinyl, homopiperidinyl and azetidinyl.

In an embodiment of the invention, when n is 1 and Ar¹ is a group (I) and Ar² is phenyl substituted by XR¹⁰ in a position para to Ar¹ and X is CH₂, then R¹⁰ is not a 2,4-dioxothiazolyl group; and when n is 1 and Ar¹ is a group (II) and Ar² is phenyl substituted by MC₁₋₆ alkylCO₂H in a position para to Ar¹, then M does not represent a bond.

In a further aspect of the present invention, there is provided a compound of general formula (I), or a pharmaceutically acceptable salt thereof,

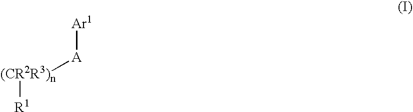

wherein Ar¹ represents a group

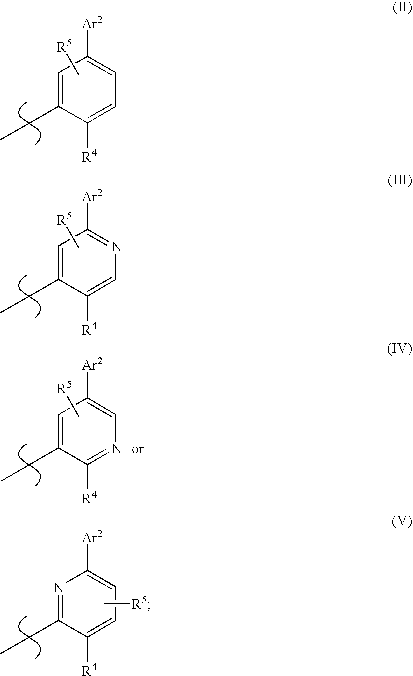

A represents C(O)NH or NHC(O);
R¹ represents phenyl or a 3- to 9-membered aliphatic carbocyclic ring, which phenyl or aliphatic carbocyclic ring can be optionally substituted by at least one substituent independently selected from halogen, hydroxyl, C₁₋₆ alkoxy and a C₁₋₆ alkyl group which C₁₋₆ alkyl group can be optionally substituted by at least one substituent independently selected from halogen and hydroxyl;
n is 0, 1, 2 or 3;
within each grouping, CR²R³, R² and R³ each independently represent hydrogen, or a C₁₋₆ alkyl group, or R² and R³ together with the carbon atom to which they are both attached form a 3- to 6-membered cycloalkyl ring;
one of R⁴ and R⁵ represents halogen, nitro, NR⁶R⁷, hydroxyl, or a C₁₋₆ alkyl group optionally substituted by at least one halogen, and the other of R⁴ and R⁵ represents hydrogen;
Ar² represents phenyl substituted by at least one substituent independently selected from carboxyl, MC₁₋₆ alkylCO₂H and NR¹⁷R¹⁸,
or Ar² represents a 5- or 6-membered heteroaromatic ring selected from thienyl, furanyl, imidazolyl, thiazolyl, oxazolyl, isothiazolyl, isoxazolyl, pyridyl, pyridazinyl, pyrimidinyl and pyrazinyl, which phenyl or heteroaromatic ring is substituted by at least one substituent independently selected from carboxyl, MC₁₋₆ alkylCO₂H and NR¹⁹R²⁰;
M represents a bond, oxygen, S(O)₀₋₂ or NR¹¹;
R⁶ and R⁷ each independently represent a hydrogen atom or a C₁₋₆ alkyl group;
R¹¹ represents a hydrogen atom or a C₁₋₆ alkyl group;
R¹⁷ and R¹⁸ together with the nitrogen atom to which they are attached form a 6-membered saturated heterocyclic ring, which heterocyclic ring is substituted with at least one substituent independently selected from carboxyl and MC₁₋₆ alkylCO₂H; and
R¹⁹ and R²⁰ together with the nitrogen atom to which they are attached form a 6-membered saturated heterocyclic ring, which heterocyclic ring is substituted with at least one substituent independently selected from carboxyl and MC₁₋₆ alkylCO₂H.

In a still further aspect of the present invention, there is provided a compound of general formula (I), or a pharmaceutically acceptable salt thereof,

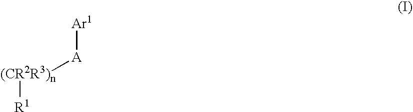

wherein Ar¹ represents a group

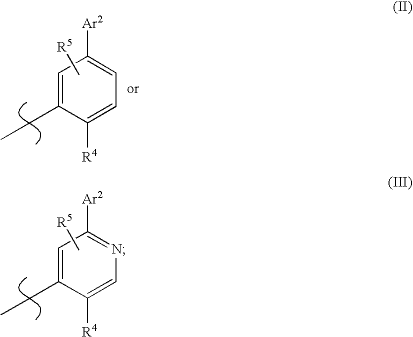

A represents NHC(O);
R¹ represents phenyl or a 3- to 9-membered aliphatic carbocyclic ring, which phenyl or aliphatic carbocyclic ring can be optionally substituted by at least one substituent independently selected from halogen, hydroxyl and a C₁₋₄ alkyl group which C₁₋₄ alkyl group can be optionally substituted by hydroxyl;
n is 0, 1 or 2;
within each grouping, CR²R³, R² and R³ each independently represent hydrogen, or a C₁₋₄ alkyl group;
one of R⁴ and R⁵ represents halogen, nitro, NR⁶R⁷, hydroxyl or a C₁₋₆ alkyl group optionally substituted by at least one halogen, and the other of R⁴ and R⁵ represents hydrogen;
Ar² represents phenyl substituted by at least one substituent independently selected from carboxyl and NR¹⁷R¹⁵,
or Ar² represents pyridyl substituted by at least one substituent independently selected from carboxyl and NR¹⁹R²⁰,
R⁶ and R⁷ each independently represent a hydrogen atom or a C₁₋₆ alkyl group;
R¹⁷ and R¹⁸ together with the nitrogen atom to which they are attached form a 6-membered saturated heterocyclic ring, which heterocyclic ring is substituted with at least one substituent independently selected from carboxyl and C₁₋₆ alkylCO₂H; and
R¹⁹ and R²⁰ together with the nitrogen atom to which they are attached form a 6-membered saturated heterocyclic ring, which heterocyclic ring is substituted with at least one substituent independently selected from carboxyl and C₁₋₆ alkylCO₂H.

In an embodiment of the invention, the compound of formula (I) is selected from
4′-Chloro-3′-[[[2-(2-chlorophenyl)ethyl]amino]carbonyl]-[1,1′-biphenyl]-2-carboxylic acid,
4′-Chloro-3′-[[[(1-hydroxycycloheptyl)methyl]amino]carbonyl]-[1,1′-biphenyl]-2-carboxylic acid,
4′-Chloro-3′-[[(cyclohexylmethyl)amino]carbonyl]-[1,1′-biphenyl]-2-carboxylic acid,
4′-Chloro-3′-[[[(2S)-2-phenylpropyl]amino]carbonyl]-[1,1′-biphenyl]-2-carboxylic acid,
4′-Chloro-3′-[[[[(1S,2R,5S)-6,6-dimethylbicyclo[3.1.1]hept-2-yl]methyl]amino]carbonyl]-[1,1′-biphenyl]-2-carboxylic acid,
4′-Chloro-3′-[[(cycloheptylmethyl)amino]carbonyl]-[1,1′-biphenyl]-2-carboxylic acid,
4′-Chloro-3′-[[[(1-hydroxycyclohexyl)methyl]amino]carbonyl]-[1,1′-biphenyl]-2-carboxylic acid,
4′-Chloro-3′-[[[[cis-2-hydroxycycloheptyl]methyl]amino]carbonyl]-[1,1′-biphenyl]-2-carboxylic acid,
4′-Chloro-3′-[[(2-cyclohexylethyl)amino]carbonyl]-[1,1′-biphenyl]-2-carboxylic acid,
3-[4-Chloro-3-[[(cycloheptylmethyl)amino]carbonyl]phenyl]-2-pyridinecarboxylic acid,
3-[4-Chloro-3-[[(2-cyclohexylethyl)amino]carbonyl]phenyl]-2-pyridinecarboxylic acid
4′-Chloro-3′-[[[(1R)-1-cyclohexylethyl]amino]carbonyl]-[1,1′-biphenyl]-2-carboxylic acid,
4′-Chloro-3′-[[[(1-methylcycloheptyl)methyl]amino]carbonyl]-[1,1′-biphenyl]-2-carboxylic acid,
4′-Chloro-3′-[[[[1-(hydroxymethyl)cycloheptyl]methyl]amino]carbonyl]-[1,1′-biphenyl]-2-carboxylic acid,
3-[4-Chloro-3-[[[(1-hydroxycycloheptyl)methyl]amino]carbonyl]phenyl]-2-pyridinecarboxylic acid,
3′-[[(Cycloheptylmethyl)amino]carbonyl]-4′-methyl-[1,1′-biphenyl]-2-carboxylic acid,
1-[3-[3-[[(Cycloheptylmethyl)amino]carbonyl]-4-methylphenyl]-2-pyridinyl]-4-piperidinecarboxylic acid,
3-Chloro-6-[3-[[(cycloheptylmethyl)amino]carbonyl]-4-methylphenyl]-2-pyridinecarboxylic acid,
5-Chloro-2-[4-chloro-3-[[(2-cyclohexylethyl)amino]carbonyl]phenyl]-3-pyridinecarboxylic acid,
5-Chloro-2-[4-chloro-3-[[(cycloheptylmethyl)amino]carbonyl]phenyl]-3-pyridinecarboxylic acid,
5-Chloro-2-[4-chloro-3-[[[(1-hydroxycycloheptyl)methyl]amino]carbonyl]phenyl]-3-pyridinecarboxylic acid,
3-Chloro-6-[4-chloro-3-[[(2-cyclohexylethyl)amino]carbonyl]phenyl]-2-pyridinecarboxylic acid,
3-Chloro-6-[4-chloro-3-[[(cycloheptylmethyl)amino]carbonyl]phenyl]-2-pyridinecarboxylic acid,
3-Chloro-6-[4-chloro-3-[[[(1-hydroxycycloheptyl)methyl]amino]carbonyl]phenyl]-2-pyridinecarboxylic acid,
1-[3-[4-Chloro-3-[[(2-cyclohexylethyl)amino]carbonyl]phenyl]-2-pyridinyl]-4-piperidinecarboxylic acid,
1-[3-[4-Chloro-3-[[(cycloheptylmethyl)amino]carbonyl]phenyl]-2-pyridinyl]-4-piperidinecarboxylic acid,
1-[3-[4-Chloro-3-[[[(1-hydroxycycloheptyl)methyl]amino]carbonyl]phenyl]-2-pyridinyl]-4-piperidinecarboxylic acid,
   or a pharmaceutically acceptable salt thereof.

The present invention further provides a process for the preparation of a compound of formula (I) as defined above, or a pharmaceutically acceptable salt thereof, which comprises:

(a) reacting a compound of formula

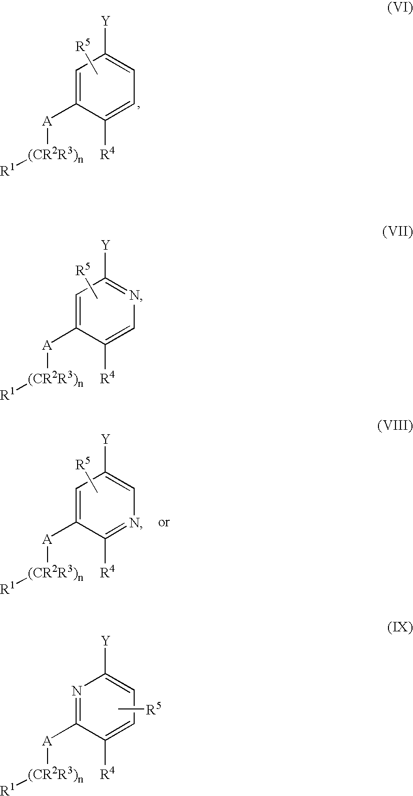

with a compound of formula

Z-Ar² (X)

wherein one of Y and Z represents a displaceable group such as a metallic, organometallic or organosilicon group (e.g. copper, lithium, an organoboron group such as B(OH)₂, B(OⁱPr)₂, BEt₂ or a boronic acid pinacol cyclic ester, or an organotin group such as SnMe₃ or SnBu₃, an organosilicon group such as Si(Me)F₂, an organoaluminium group such as AlEt₂, an organomagnesium group such as MgCl, MgBr or MgI, or an organozinc group such as ZnCl, ZnBr or ZnI) and the other of Y and Z represents a leaving group such as a halogeno or sulphonyloxy group (e.g. a chloro, bromo, iodo, trifluoromethanesulphonyloxy, methanesulphonyloxy or paratoluenesulphonyloxy group) and Ar², R¹, R², R³, n, A, R⁴ and R⁵ are as defined in formula (I); or
(b) when Ar² is substituted by carboxyl, reacting a compound of formula (VI)-(IX) as defined in (a) above with a compound of formula

Z-Ar^{2a}—CO₂R¹² (XI)

wherein Z is as defined in formula (X), Ar^{2a} represents a phenyl or 5- or 6-membered heteroaromatic ring comprising from 1 to 2 heteroatoms independently selected from nitrogen, oxygen and sulphur, and R¹² is a C₁₋₆ alkyl group, followed by reaction with a base such as sodium hydroxide or lithium hydroxide in a solvent such as water or methanol, at a temperature in the range 0-150° C., or followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or
(c) when Ar² is substituted by carboxyl, reacting a compound of formula (VI)-(IX) as defined in (a) above with a compound of formula

Z-Ar^{2b}—CN (XII)

wherein Z is as defined in formula (X), and Ar^{2b} represents a phenyl or 5- or 6-membered heteroaromatic ring comprising from 1 to 2 heteroatoms independently selected from nitrogen, oxygen and sulphur, followed by reaction with a base such as sodium hydroxide or lithium hydroxide in a solvent such as water or methanol, at a temperature in the range 0-150° C., or followed by reaction with an acid such as hydrochloric acid in a solvent such as water, at a temperature in the range 0-150° C.; or
(d) when R⁸ represents CN, C₁₋₆alkoxycarbonyl, C₁₋₆ alkylaminosulphonyl, or (di)-C₁₋₆ alkylaminosulphonyl, reacting a compound of formula (VI)-(IX) as defined in (a) above with a compound of formula

L¹-Ar^{2c}-Z (XIII)

wherein L¹ represents a leaving group such as a halogeno or sulphonyloxy group (e.g. a chloro, bromo, iodo, trifluoromethanesulphonyloxy, methanesulphonyloxy or paratoluenesulphonyloxy group), Ar^{2c} represents a phenyl, 5- or 6-membered heteroaromatic ring comprising from 1 to 2 heteroatoms independently selected from nitrogen, oxygen and sulphur, and Z is as defined in formula (X), followed by reaction with a compound of formula

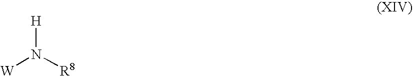

wherein W represents a hydrogen or a metallic group, for example sodium, and R⁸ is as defined in formula (I); or
(e) when Ar² is substituted by carboxyl, reacting a compound of formula (VI)-(IX) as defined in (a) above with a compound of formula (XIII) as defined in (d) above, followed by reaction with a suitable source of cyanide (e.g. sodium cyanide, potassium cyanide, copper cyanide or zinc cyanide), followed by reaction with a base such as sodium hydroxide or lithium hydroxide in a solvent such as water or methanol, at a temperature in the range 0-150° C., or followed by reaction with an acid such as hydrochloric acid in a solvent such as water, at a temperature in the range 0-150° C.; or
(f) when Ar² is substituted by carboxyl, reacting a compound of formula (VI)-(IX) as defined in (a) above with a compound of formula (XIII) as defined in (d) above, followed by reaction with carbon monoxide and an alcohol in the presence of a suitable catalyst, for example a palladium catalyst, followed by reaction with a base such as sodium hydroxide or lithium hydroxide in a solvent such as water or methanol, at a temperature in the range 0-150° C.; or
(g) reacting a compound of formula

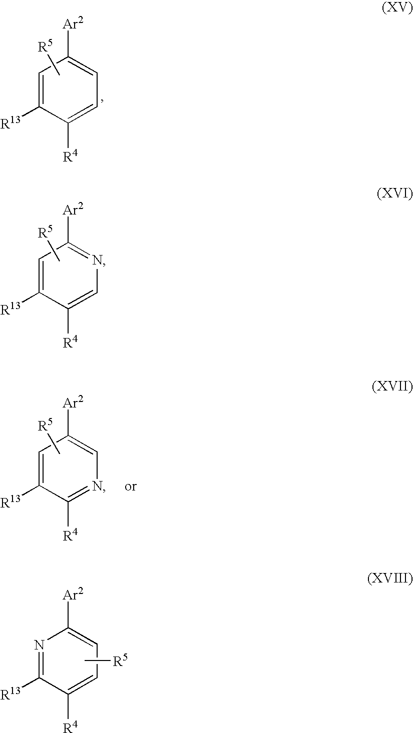

with a compound of formula

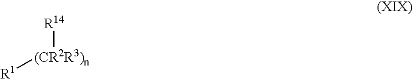

wherein one of R¹³ and R¹⁴ represents NH₂ and the other of R¹³ and R¹⁴ represents CO₂H, COBr or COCl, and R¹, R², R³, n, R⁴, R⁵ and Ar² are as defined in formula (I); or
(h) reacting a compound of formula

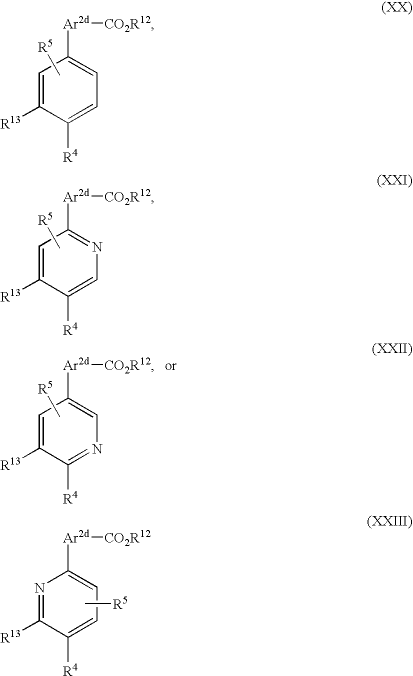

with a compound of formula (XIX) as defined in (g) above, wherein Ar^{2d} represents a phenyl or 5- or 6-membered heteroaromatic ring comprising from 1 to 2 heteroatoms independently selected from nitrogen, oxygen and sulphur, R¹² is as defined in formula (XI), R⁴ and R⁵ are as defined in formula (I), and R¹³ is as defined in formula (XV)-(XVIII), followed by reaction with a base such as sodium hydroxide or lithium hydroxide in a solvent such as water or methanol, at a temperature in the range 0-150° C., or followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.;
(i) when R¹⁹ and R²⁰ together with the nitrogen to which they are attached form a 3- to 8-membered saturated heterocyclic ring, which heterocyclic ring is substituted by carboxyl, reacting a compound of formula (VI)-(IX) as defined in (a) above wherein Y represents a displaceable group such as an organoboron group (e.g. B(OH)₂, B(OⁱPr)₂, BEt₂ or a boronic acid pinacol cyclic ester), with a compound of formula

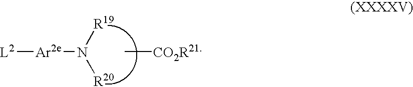

wherein R²¹ represents a C₁₋₆alkyl group, Ar^{2e} represents a 5- or 6-membered heteroaromatic ring comprising from 1 to 2 heteroatoms independently selected from nitrogen, oxygen and sulphur, L² represents a leaving group such as a halogeno or sulphonyloxy group (e.g. a chloro, bromo, iodo, trifluoromethanesulphonyloxy, methanesulphonyloxy or paratoluenesulphonyloxy group), and R¹⁹ and R²⁰ are as defined in formula (I), optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or
(j) when R¹⁹ and R²⁰ together with the nitrogen to which they are attached form a 3- to 8-membered saturated heterocyclic ring, which heterocyclic ring is substituted by carboxyl, reacting a compound of formula (XIX) as defined in (g) above, with a compound of formula

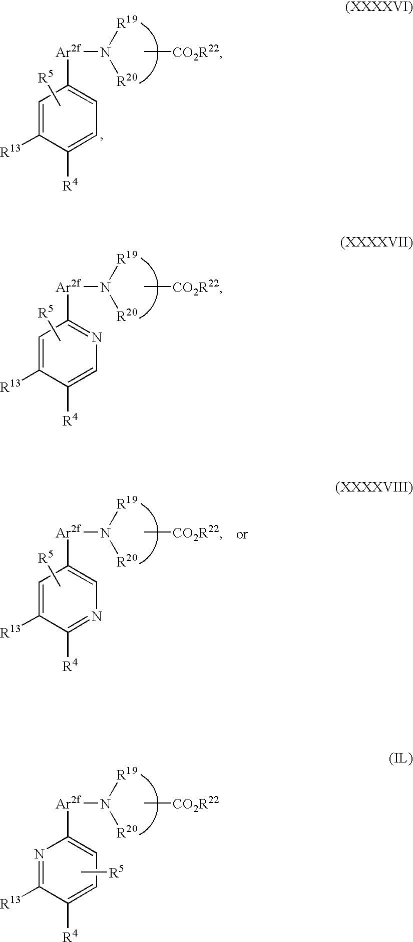

wherein Ar^{2f} represents a 5- or 6-membered heteroaromatic ring comprising from 1 to 2 heteroatoms independently selected from nitrogen, oxygen and sulphur, R²² is a C₁₋₆alkyl group, R⁴, R⁵, R¹⁹ and R²⁰ are as defined in formula (I), and R¹³ is as defined in formula (XV)-(XVIII), followed by reaction with a base such as sodium hydroxide or lithium hydroxide in a solvent such as water or methanol, at a temperature in the range 0-150° C., or followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; and optionally after (a), (b), (c), (d), (e), (f), (g), (h), (i) or (j), carrying out one or more of the following:
converting the compound to a further compound of the invention
forming a pharmaceutically acceptable salt of the compound.

In formula (XI), (XII), (XIII), (XX), (XXI), (XXII) and (XXIII) above, Ar^{2a}, Ar^{2b} Ar^{2c} and Ar^{2d}, which independently represent a phenyl or 5- or 6-membered heteroaromatic ring, can further be optionally substituted with at least one substituent, which at least one substituent is as defined in formula (I) for further optional substituents on Ar².

In formula (XXXXV), (XXXXVI), (XXXXVII), (XXXXVIII) and (IL) above, Ar^{2e} and Ar^{2f}, which independently represent a 5- or 6-membered heteroaromatic ring, can further be optionally substituted with at least one substituent, which at least one substituent is as defined in formula (I) for further optional substituents on Ar².

In processes (a), (b), (c), (d), (e), (f) and (i), the coupling reaction is conveniently carried out in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C.

In process (d), the displacement reaction may be carried out in the presence of a suitable base, for example potassium tert-butoxide, sodium hydride, potassium carbonate or caesium carbonate, optionally in the presence of a suitable catalyst, for example a palladium catalyst such as tetrakis(triphenylphosphine)palladium(0), palladium(II) chloride, palladium(II) bromide, palladium(II) acetate, dichlorobis(triphenylphosphine)palladium(II) or tris(dibenzylideneacetone)palladium(0), or a copper catalyst such as copper(I) iodide, optionally in the presence of a suitable ligand, for example 1,1′-bis(diphenylphosphino)ferrocene, 9,9-dimethyl-4,5-bis(diphenylphosphino)xanthene or 2-dicyclohexylphosphino-2′-(N,N-dimethylamino)biphenyl, in the presence of a suitable solvent, for example 1-methyl-2-pyrrolidinone, 1,4-dioxane, 1,2-dimethoxyethane, tetrahydrofuran or acetonitrile, and at a temperature in the range 10 to 250° C., preferably in the range 60 to 150° C.

In process (e), the displacement reaction may be carried out in the presence of a suitable source of cyanide, for example sodium cyanide, potassium cyanide, copper cyanide or zinc cyanide, optionally in the presence of a suitable catalyst, for example a palladium catalyst such as tetrakis(triphenylphosphine)palladium(0) or palladium(II) acetate, in the presence of a suitable solvent, for example N,N-dimethylformamide, 1-methyl-2-pyrrolidinone or dimethylsulfoxide, and at a temperature in the range 10-250° C., preferably in the range 60 to 150° C.

In process (f), the carbonylation reaction may be carried out in the presence of an alcohol such as butanol, propanol, ethanol or methanol, in the presence of a catalyst such as tetrakis(triphenylphosphine)palladium(0), palladium(II) chloride, palladium(II) bromide, palladium(II) acetate, dichlorobis(triphenylphosphine)palladium (II) or [1,1′-bis(diphenylphosphino)ferrocene]palladium(II) chloride, optionally in the presence of a ligand such as triphenylphosphine or 1,3-bis(diphenylphosphino)propane, in the presence of a suitable base, for example triethylamine, optionally in the presence of a co-solvent, for example 1-methyl-2-pyrrolidinone or N,N-dimethylformamide, and at a temperature in the range 10-150° C.

In processes (g), (h) and (j), the amide coupling reaction may be carried out in the presence of a suitable coupling reagent, such as 1,1′-carbonyldiimidazole or dicyclohexylcarbodiimide and 1-hydroxybenzotriazole, in the presence of a base such as triethylamine, N-methylmorpholine, diisopropylethylamine or potassium carbonate, in a solvent such as dichloromethane, N-methylpyrrolidinone, N—N-dimethylformamide or tetrahydrofuran, and at a temperature in the range 0-150° C.

It will be appreciated by those skilled in the art that in the processes of the present invention certain functional groups such as hydroxyl, carboxyl or amino groups in the starting reagents or intermediate compounds may need to be protected by protecting groups. Thus, the preparation of the compounds of formula (I) may involve at a certain stage the removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994).

The compounds of formula (I) above may be converted to a pharmaceutically acceptable salt thereof. Where the compound is sufficiently acidic, suitable salts include base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Where the compound is sufficiently basic, suitable salts include acid addition salts such as a hydrochloride, hydrobromide, phosphate, acetate, fumarate, maleate, tartrate, citrate, oxalate, methanesulphonate or p-toluenesulphonate salt. There may be more than one cation or anion depending on the number of charged functions and the valency of the cations or anions. Other pharmaceutically acceptable salts, as well as prodrugs such as pharmaceutically acceptable esters and pharmaceutically acceptable amides may be prepared using conventional methods.

Compounds of formula (VI)-(IX), wherein Y represents an organoboron group such as B(OH)₂ or B(OⁱPr)₂, may be prepared by reacting compounds of formula (VI)-(IX), wherein Y represents a displaceable group such as bromo or iodo, with suitable organometallic reagents, for example methyllithium and tert-butyllithium, in the presence of a trialkylborate, e.g. triisopropylborate, in the presence of a suitable solvent such as tetrahydrofuran, and at a temperature in the range −100° C. to 30° C., and optionally followed by hydrolysis of the boronate ester by reaction with an acid such as ammonium chloride in a solvent such as water or tetrahydrofuran, at a temperature in the range 0-150° C.

Alternatively, compounds of formula (VI)-(IX), wherein Y represents an organoboron group such as B(OH)₂ or a boronic acid pinacol cyclic ester may be prepared by reacting compounds of formula (VI)-(IX), wherein Y represents a displaceable group such as a halogeno or sulphonyloxy group, for example a chloro, bromo, iodo, trifluoromethanesulphonyloxy, methanesulphonyloxy or paratoluenesulphonyloxy group, with a suitable diboron reagent, e.g. bis(pinacolato)diboron, in the presence of a catalyst, for example palladium acetate or [1,1′-bis(diphenylphosphino)ferrocene]palladium(II) chloride, in the presence of a base such as potassium acetate or tripotassium phosphate, in the presence of a suitable solvent, e.g. dimethylsulphoxide, N,N-dimethylformamide, 1,4-dioxane or tetrahydrofuran, and at a temperature in the range 25-250° C., and optionally followed by hydrolysis of the boronate ester by reaction with an acid such as ammonium chloride in a solvent such as water or tetrahydrofuran, at a temperature in the range 0-150° C.

Compounds of formula (VI)-(IX), wherein Y represents a leaving group such as a halogeno or sulphonyloxy group, may conveniently be prepared by reacting a compound of general formula (XIX) with a compound of general formula

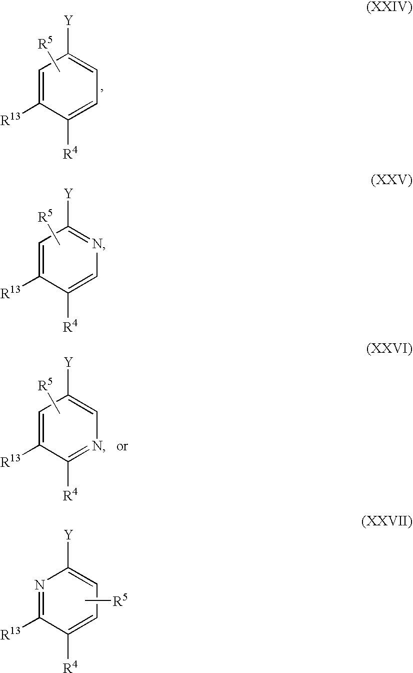

wherein Y represents a leaving group such as a halogeno or sulphonyloxy group as defined in formula (VI)-(IX), R⁴ and R⁵ are as defined in formula (I), and R¹³ is as defined in formula (XV)-(XVIII), optionally in the presence of suitable coupling reagents such as 1,1′-carbonyldiimidazole or dicyclohexylcarbodiimide and 1-hydroxybenzotriazole.

Compounds of formula (XV)-(XVIII) where R¹³ is a carboxyl group may be prepared by reacting a compound of general formula

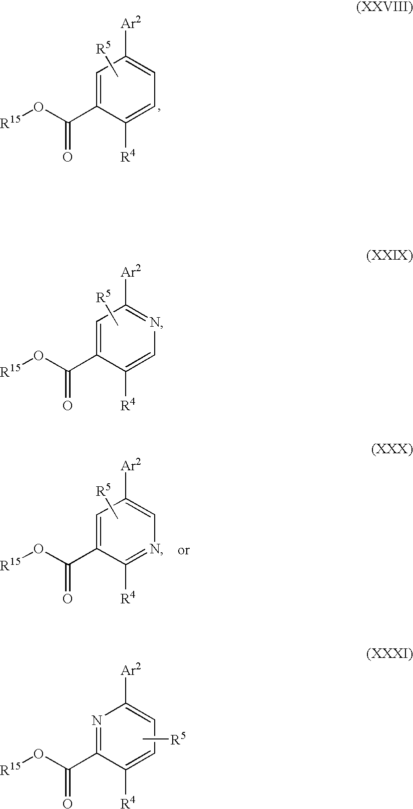

wherein R¹⁵ is a C₁₋₆ alkyl group, and Ar², R⁴ and R⁵ are as defined in formula (I), with a base such as sodium hydroxide or lithium hydroxide in a solvent such as water or methanol, at a temperature in the range 0-150° C., or with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.

Compounds of formula (XX)-(XXIII) where R¹³ is a carboxyl group may be prepared by reacting a compound of general formula

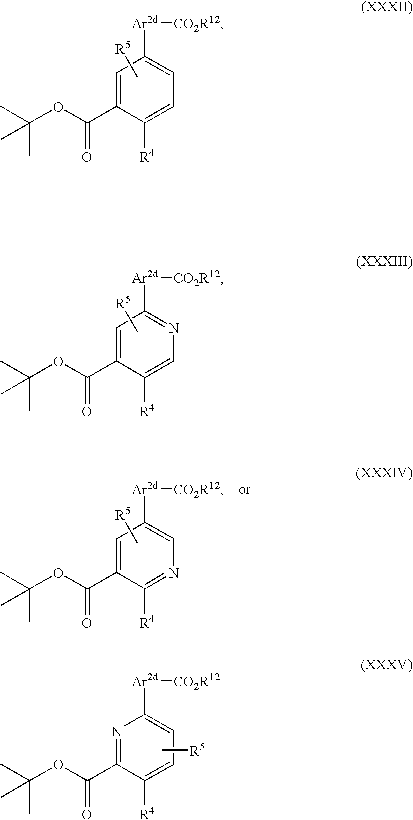

wherein Ar^{2d} is as defined in formula (XX)-(XXIII), R¹² is as defined in formula (XI), and R⁴ and R⁵ are as defined in formula (I), with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.

Compounds of formula (XXVIII)-(XXXI) may be prepared by reacting a compound of general formula

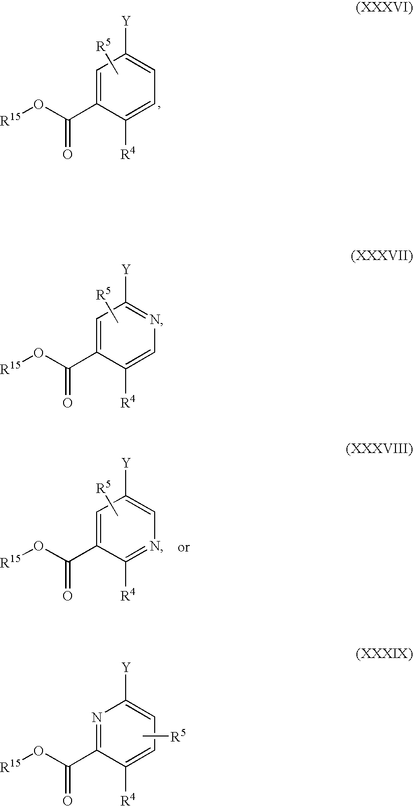

with a compound of formula (X) as defined in (a) above, wherein Y is as defined in formula (VI)-(IX), R¹⁵ is as defined in formula (XXVIII)-(XXXI), and R⁴ and R⁵ are as defined in formula (I), in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C.

Compounds of formula (XXXII)-(XXXV) may be prepared by reacting a compound of formula (XXXVI)-(XXXIX), wherein R¹⁵ is a tert-butyl group, with a compound of formula

Z-Ar^{2d}—CO₂R¹² (XXXX)

wherein Z is as defined in formula (X), Ar^{2d} is as defined in formula (XX)-(XXIII) and R¹² is as defined in formula (XI), in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C.

Compounds of formula (XXXVI)-(XXXIX), wherein Y represents an organoboron group such as B(OH)₂ or B(OⁱPr)₂, may be prepared by reacting compounds of formula (XXXVI)-(XXXIX), wherein Y represents a displaceable group such as bromo or iodo, with suitable organometallic reagents, for example methyllithium and tert-butyllithium, in the presence of a trialkylborate, e.g. triisopropylborate, in the presence of a suitable solvent such as tetrahydrofuran, and at a temperature in the range −100° C. to 30° C., and optionally followed by hydrolysis of the boronate ester by reaction with an acid such as ammonium chloride in a solvent such as water or tetrahydrofuran, at a temperature in the range 0-150° C.

Alternatively, compounds of formula (XXXVI)-(XXXIX), wherein Y represents an organoboron group such as B(OH)₂ or a boronic acid pinacol cyclic ester may be prepared by reacting compounds of formula (XXXVI)-(XXXIX), wherein Y represents a displaceable group such as a halogeno or sulphonyloxy group, for example a chloro, bromo, iodo, trifluoromethanesulphonyloxy, methanesulphonyloxy or paratoluenesulphonyloxy group, with a suitable diboron reagent, e.g. bis(pinacolato)diboron, in the presence of a catalyst, for example palladium acetate or [1,1′-bis(diphenylphosphino)ferrocene]palladium(II) chloride, in the presence of a base such as potassium acetate or tripotassium phosphate, in the presence of a suitable solvent, e.g. dimethylsulphoxide, 1,4-dioxane or tetrahydrofuran, and at a temperature in the range 25-250° C., and optionally followed by hydrolysis of the boronate ester by reaction with an acid such as ammonium chloride in a solvent such as water or tetrahydrofuran, at a temperature in the range 0-150° C.

Compounds of formula (XXXVI)-(XXXIX), wherein Y represents a leaving group such as a halogeno or sulphonyloxy group, may conveniently be prepared by reacting a compound of formula

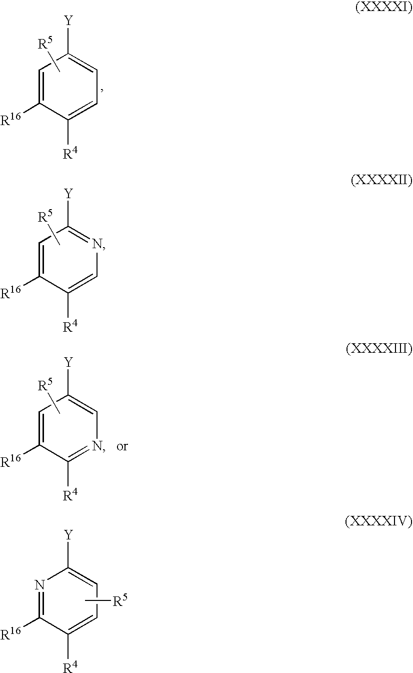

wherein R¹⁶ represents CO₂H, COBr or COCl, Y is a leaving group as defined in formula (VI)-(IX), and R⁴ and R⁵ are as defined in formula (I), with an alcohol or a metal alkoxide such as potassium tert-butoxide, optionally in the presence of suitable reagents such as dicyclohexylycarbodiimide and 4-dimethylaminopyridine.

Compounds of formula (XXXXV) may be conveniently prepared by reacting a compound of formula

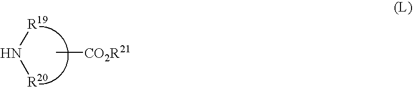

wherein R²¹ is as defined as in formula (XXXXV) and R¹⁹ and R²⁰ are as defined in formula (I), with a compound of formula

L²-Ar^{2e}-L³ (LI)

wherein L³ represents a leaving group such as a halogeno or sulphonyloxy group (e.g. a chloro, bromo, iodo, trifluoromethanesulphonyloxy, methanesulphonyloxy or paratoluenesulphonyloxy group), and L² and Ar^{2e} are as defined in formula (XXXXV).

Compounds of formula (XXXXVI)-(IL) wherein R¹³ represents a carboxyl group may be prepared by reacting a compound of general formula

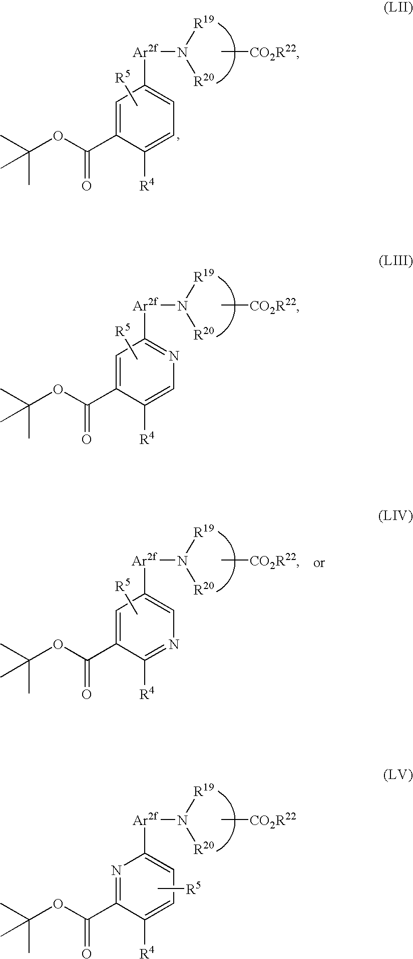

wherein Ar^{2f} and R²² are as defined in formula (XXXXVI)-(IL), and R⁴, R⁵, R¹⁹ and R²⁰ are as defined in formula (I), with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.

Compounds of formula (LII)-(LV) may be prepared by reacting a compound of formula (XXXVI)-(XXXIX), wherein Y represents a displaceable group such as an organoboron group (e.g. B(OH)₂, B(OⁱPr)₂, BEt₂ or a boronic acid pinacol cyclic ester) and R¹⁵ is a tert-butyl group, with a compound of formula (XXXXV), in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C.

Compounds of formula (X), (XI), (XII), (XIII), (XIV), (XIX), (XXIV), (XXV), (XXVI), (XXVII), (XXXX), (XXXXI), (XXXXII), (XXXXIII), (XXXXIV), (L) and (LI) are either commercially available, are known in the literature or may be prepared easily using known techniques.

A compound of the invention, or a pharmaceutically acceptable salt thereof may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS. Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention provides a compound of formula (I), or a pharmaceutically acceptable salt thereof, as hereinbefore defined for use in therapy.

In another aspect, the invention provides the use of a compound of formula (I), or a pharmaceutically acceptable salt thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

An embodiment of the invention provides the use of a compound of formula (I) as defined herein above, or a pharmaceutically acceptable salt thereof, in the manufacture of a medicament for use in the treatment of rheumatoid arthritis.

An embodiment of the invention provides the use of a compound of formula (I) as defined herein above, or a pharmaceutically acceptable salt thereof, in the manufacture of a medicament for use in the treatment of osteoarthritis.

An embodiment of the invention provides the use of a compound of formula (I) as defined herein above, or a pharmaceutically acceptable salt thereof, in the manufacture of a medicament for use in the treatment of asthma or chronic obstructive pulmonary disease.

An embodiment of the invention provides the use of a compound of formula (I) as defined herein above, or a pharmaceutically acceptable salt thereof, in the manufacture of a medicament for use in the treatment of atherosclerosis.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt thereof, as hereinbefore defined to a patient.

For all the above-mentioned therapeutic uses the dosage administered will, of course, vary with the compound employed, the mode of administration, the treatment desired and the disorder indicated. The daily dosage of the compound of formula (I)/salt (“active ingredient”) may be in the range from 0.001 mg/kg to 30 mg/kg.

The compounds of formula (I) and pharmaceutically acceptable salts thereof may be used on their own but will generally be administered in the form of a pharmaceutical composition in which the formula (I) compound/salt/solvate (“active ingredient”) is in association with a pharmaceutically acceptable adjuvant, diluent or carrier. Depending on the mode of administration, the pharmaceutical composition will preferably comprise from 0.05 to 99% w (percent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Thus, the present invention also provides a pharmaceutical composition comprising a compound of formula (I), or a pharmaceutically acceptable salt thereof, as hereinbefore defined in association with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxifylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer'drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B1- or B2 receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK1 or NK3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; or (xxvii) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an estrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples. In the examples the NMR spectra were measured on a Varian Unity spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluant. Microwave reactions were performed in a CEM Discover single mode microwave. In the following examples all compounds were named using the Chemical Abstracts Service Index Name function within the ACD/Name software package.

### EXAMPLE 1

### 4′-Chloro-3′-[[[2-(2-chlorophenyl)ethyl]amino]carbonyl]-[1,1′-biphenyl]-2-carboxylic acid

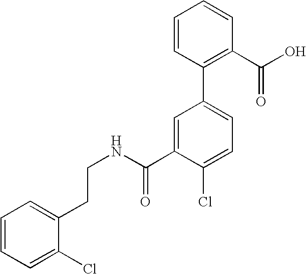

### a) [4-Chloro-3-[[[2-(2-chlorophenyl)ethyl]amino]carbonyl]phenyl]-boronic acid

Methyllithium (1.6M in diethyl ether, 3.0 mL) was added to a stirred solution of 5-bromo-2-chloro-N-[2-(2-chlorophenyl)ethyl]-benzamide (Prepared as described in WO2003042191) (1.5 g) in tetrahydrofuran (40 mL) at −78° C. After 10 minutes, triisopropyl borate (4.8 mL) was added, followed by tert-butyllithium (1.7M in pentane, 5.2 mL). After stirring at −78° C. for 2 hours the mixture was allowed to warm to −300° C., saturated aqueous ammonium chloride (40 mL) was cautiously added and the mixture was allowed to warm to room temperature over 16 hours. Ethyl acetate (100 mL) was added, the layers were separated and the aqueous fraction was extracted with ethyl acetate (2×50 mL). The combined organic fractions were dried (MgSO₄), filtered and concentrated in vacuo to yield the sub-title compound as a colourless solid (1.3 g).

MS: APCI(+ve) 338 (M+H⁺).

### b) 4′-Chloro-3′-[[[2-(2-chlorophenyl)ethyl]amino]carbonyl]-[1,1′-biphenyl]-2-carboxylic acid, methyl ester

A mixture of [4-chloro-3-[[[2-(2-chlorophenyl)ethyl]amino]carbonyl]phenyl]-boronic acid (600 mg) (Example 1 (a)), 2-bromobenzoic acid, methyl ester (865 mg), potassium carbonate (800 mg) and dichlorobis(triphenylphosphine)palladium (II) (100 mg) in 1,4-dioxane (9 mL)/water (9 mL) was heated at 65° C. under a nitrogen atmosphere for 3 hours. The products were filtered through diatomaceous earth, washing with methanol (3×30 mL). The solvent was removed in vacuo and the residue was purified by chromatography (SiO₂, dichloromethane:methanol 99:1 as eluant) to yield the sub-title compound as a solid (230 mg).

MS: APCI(+ve) 430 (M+H⁺).

### c) 4′-Chloro-3′-[[[2-(2-chlorophenyl)ethyl]amino]carbonyl]-[1,1′-biphenyl]-2-carboxylic acid

A mixture of 4′-chloro-3′-[[[2-(2-chlorophenyl)ethyl]amino]carbonyl]-[1,1′-biphenyl]-2-carboxylic acid, methyl ester (Example 1 (b)) (230 mg), methanol (1 mL) and aqueous sodium hydroxide (6M, 1.0 mL) was heated in a microwave at 65° C. for 30 minutes. Purification by chromatography (SiO₂, dichloromethane:methanol 99:1, then dichloromethane:methanol 97:3 as eluant), then by Varian NH₂ cartridge using methanol (100 mL) and then 1% trifluoroacetic acid in methanol (100 mL) as eluant, and then by RP-HPLC, acetonitrile:aqueous ammonium acetate, Symmetry) gave the title compound as a solid (60 mg).

MS: APCI(+ve) 414 (M+H⁺).

m.p. 136-140° C. dec.

¹HNMR (400 MHz, d₆-DMSO) δ 8.59 (1H, t), 7.75 (1H, d), 7.58 (1H, t), 7.51-7.21 (9H, m), 3.49 (2H, td), 2.97 (2H, t).

### EXAMPLE 2

### 4′-Chloro-3′-[[[(1-hydroxycycloheptyl)methyl]amino]carbonyl]-[1,1′-biphenyl]-2-carboxylic acid

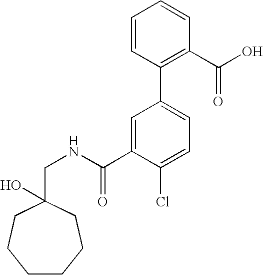

### a) 1-[(Trimethylsilyl)oxy]-cycloheptanecarbonitrile

The sub-title compound was prepared according to the method of J. Med. Chem., 1981, 24, 7-12. Trimethylsilylcyanide (8.8 g, 12 mL) was added over 30 minutes to a stirred mixture of cycloheptanone (10 g) and zinc iodide (0.01 g) at 0° C. under nitrogen. The mixture was allowed to warm to room temperature over 72 hours and the sub-title compound (18.8 g) was used without purification.

¹H NMR (400 MHz, CDCl₃) δ 1.91-1.82 (2H, m), 1.75-1.64 (2H, m), 1.53-1.26 (8H, m), 0.00 (9H, s).

### b) 1-(Aminomethyl)-cycloheptanol

The sub-title compound was prepared according to the method of J. Med. Chem., 1981, 24, 7-12. A solution of 1-[(trimethylsilyl)oxy]-cycloheptanecarbonitrile (Example 2 (a)) (5.0 g) in tetrahydrofuran (120 mL) was added to a stirred solution of lithium aluminium hydride in diethyl ether (72 mL, 1.0M) under nitrogen at room temperature over 10 minutes. The mixture was heated at 50° C. for 1 hour before cooling to 0° C. in an ice bath and was quenched by cautious addition of water (3 mL), followed by aqueous sodium hydroxide (3 mL, 15% wt/wt), followed by water (9 mL). The volatile components were removed in vacuo and the residue was partitioned between diethyl ether (100 mL) and water (50 mL). The layers were separated, the aqueous fraction was extracted with diethyl ether (2×50 mL) and the combined organic layers were concentrated to yield the sub-title compound as a liquid (3.3 g).

¹H NMR (400 MHz, CDCl₃) δ 4.61 (2H, s), 2.44 (2H, s), 1.63-1.15 (12H, m).

### c) 2-Chloro-N-[(1-hydroxycycloheptyl)methyl]-5-iodo-benzamide

To a solution of 5-iodo-2-chlorobenzoic acid (730 mg) in dichloromethane (30 mL) at 0° C. was added triethylamine (0.72 mL), 1-hydroxybenzotriazole (435 mg), 1-(3-dimethylaminopropyl)-3-ethylcarbodiimide hydrochloride (1 g), and 1-(aminomethyl)-cycloheptanol (Example 2 (b)) (407 mg). The reaction mixture was allowed to warm to room temperature and stirred under nitrogen for 16 hours. The mixture was then poured into water (30 mL). The layers were separated and the aqueous was extracted with dichloromethane (2×30 mL). The combined organics were washed with 2M aqueous hydrochloric acid (2×30 mL), saturated aqueous sodium hydrogen carbonate (30 mL) and brine (30 mL) before being dried, filtered and evaporated to give the sub-title compound as a colourless solid (900 mg).

MS: APCI(−ve) 406/409 (M−H⁺).

¹H NMR (400 MHz, d₆-DMSO) δ 8.31 (1H, t), 7.79-7.73 (2H, m), 7.28 (1H, dd), 4.24 (1H, s), 3.19 (2H, d), 1.67-1.27 (12H, m).

### d) 4′-Chloro-3′-[[[(1-hydroxycycloheptyl)methyl]amino]carbonyl]-[1,1′-biphenyl]-2-carboxylic acid, ethyl ester

To a stirred mixture of ethyl-2-(4,4,5,5-tetramethyl-1,3,2-dioxaborolan-2-yl)benzoate (204 mg) and 2-chloro-N-[(1-hydroxycycloheptyl)methyl]-5-iodo-benzamide (Example 2 (c)) (300 mg) in tetrahydrofuran (3 mL) was added a solution of potassium carbonate (204 mg) in water (3 mL) followed by bis(triphenylphosphine)palladium(II) chloride (26 mg). The mixture was stirred at room temperature for 16 hours and then concentrated. The residue was partitioned between water (100 mL) and dichloromethane (100 mL). The layers were separated and the aqueous was extracted with dichloromethane (100 mL). The combined organics were filtered through diatomaceous earth and then concentrated. Purification (SiO₂, 3:1 isohexane:ethyl acetate as the eluant) gave the sub-title compound as a solid (300 mg).

MS: APCI(+ve) 412/414 (M+H—H₂O).

¹H NMR (400 MHz, d₆-DMSO) δ 8.20 (1H, t), 7.79 (1H, dd), 7.66 (1H, td), 7.56-7.51 (2H, m), 7.47 (1H, dd), 7.38-7.33 (2H, m), 4.09 (2H, q), 3.22 (2H, d), 1.69-1.28 (12H, m), 1.05 (3H, t).

### e) 4′-Chloro-3′-[[[(1-hydroxycycloheptyl)methyl]amino]carbonyl]-[1,1′-biphenyl]-2-carboxylic acid

4′-Chloro-3′-[[[(1-hydroxycycloheptyl)methyl]amino]carbonyl]-[1,1′-biphenyl]-2-carboxylic acid, ethyl ester (Example 2 (d)) (300 mg) and methanol (1 mL) were placed in a 10 mL microwave vial. A solution of potassium hydroxide (100 mg) in water (2 mL) was added and the mixture was heated at 50° C. for 15 minutes within a CEM Discovery microwave. The mixture was evaporated and water (5 mL) was added to the residue and this was then acidified to pH 2 with 2M hydrochloric acid. The resulting solution was extracted with dichloromethane (3×20 mL). The extracts were combined and concentrated. Purification (Varian NH₂ cartridge using dichloromethane (100 mL) and then 10% acetic acid in dichloromethane (100 mL) as eluant) afforded the title compound as a solid (60 mg).

MS: APCI(+ve) 402/404 (M+H⁺).

m.p. 113-116° C.

¹H NMR (400 MHz, d₆-DMSO) δ 8.21 (1H, t), 7.71 (1H, d), 7.57-7.35 (6H, m), 3.22 (2H, d), 1.71-1.28 (12H, m).

### EXAMPLE 3

### 4′-Chloro-3′-[[(cyclohexylmethyl)amino]carbonyl]-[1,1′-biphenyl]-2-carboxylic acid

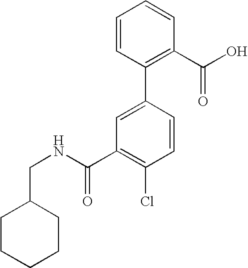

### a) 2-Chloro-5-iodo-benzoic acid, 1,1-dimethylethyl ester

N,N-Dimethylformamide (1 drop) and oxalyl chloride (4.8 mL) were added to a stirred solution of 2-chloro-5-iodobenzoic acid (5 g) in dichloromethane (20 mL) at 0° C. The reaction was allowed to warm to room temperature, stirred under nitrogen for 2 hours, and then evaporated to dryness. The residue was dissolved in tetrahydrofuran (20 mL) and cooled to 0° C. Potassium tert-butoxide (22 mL, 1 M solution in tetrahydrofuran) was added over 10 minutes. The reaction was allowed to warm to room temperature and stirred under nitrogen for 2 hours then poured into saturated aqueous sodium bicarbonate (50 mL). The layers were separated and the aqueous was extracted with diethyl ether (50 mL). The combined organics were dried, filtered and evaporated to afford the sub-title compound as an oil (5.7 g).

¹H NMR (400 MHz, d₆-DMSO) δ 7.99 (1H, d), 7.87 (1H, dd), 7.34 (1H, d), 1.54 (9H, s).

### b) 2-Chloro-5-(4,4,5,5-tetramethyl-1,3,2-dioxaborolan-2-yl)-benzoic acid, 1,1-dimethylethyl ester

A mixture of 2-chloro-5-iodo-benzoic acid, 1,1-dimethylethyl ester (Example 3 (a)) (5 g), bis(pinacolato)diboron (6 g), [1,1′-bis(diphenylphosphino)ferrocene]dichloropalladium(II) dichloromethane (600 mg) and potassium acetate (6.5 g) in N,N-dimethylformamide (50 mL) was heated to 90° C. under nitrogen for 90 minutes. The mixture was allowed to cool then diluted with 2:1 ethyl acetate: diethyl ether (250 mL) and filtered through diatomaceous earth. The filtrate was washed with water (250 mL) and brine (100 mL) then evaporated. Purification by chromatography (SiO₂, 1:1 diethyl ether:isohexane as eluant) afforded the sub-title compound as a solid (5.5 g).

MS: APCI(+ve) 282 (M-C₄H₈+H⁺).

¹H NMR (300 MHz, d₆-DMSO) δ 7.88 (1H, d), 7.76 (1H, dd), 7.56 (1H, d), 1.55 (9H, s), 1.32 (12H, s).

### c) 4′-Chloro-[1,1′-biphenyl]-2,3′-dicarboxylic acid, 3′-(1,1-dimethylethyl) 2-methyl ester

A mixture of 2-chloro-5-(4,4,5,5-tetramethyl-1,3,2-dioxaborolan-2-yl)-benzoic acid, 1,1-dimethylethyl ester (Example 3 (b)) (3.5 g), methyl-2-bromobenzoate (2.23 g), potassium carbonate (2.87 g), bis(triphenylphosphine)palladium(II) chloride (365 mg), tetrahydrofuran (20 mL) and water (20 mL) was stirred at room temperature under a nitrogen atmosphere for 16 hours. The solvent was removed in vacuo and the residue was purified by chromatography (SiO₂, 98:2 isohexane:ethyl acetate as eluant) to give the sub-title compound as a solid (2.15 g).

¹H NMR (300 MHz, d₆-DMSO) δ 7.82 (1H, dd), 7.67 (1H, td), 7.61-7.52 (3H, m), 7.49-7.43 (2H, m), 3.64 (3H, s), 1.55 (9H, s).

### d) 4′-Chloro-[1,1′-biphenyl]-2,3′-dicarboxylic acid, 2-methyl ester

Trifluoroacetic acid (3.3 mL) was added to a stirred solution of 4′-chloro-[1,1′-biphenyl]-2,3′-dicarboxylic acid, 3′-(1,1-dimethylethyl), 2-methyl ester (Example 3 (c)) (2.15 g) in dichloromethane (10 mL) and the mixture was stirred at room temperature under nitrogen for 90 minutes. The mixture was then evaporated to afford the sub-title compound as a solid (1.7 g).

¹H NMR (400 MHz, d₆-DMSO) δ 7.82 (1H, dd), 7.69-7.64 (2H, m), 7.59 (1H, d), 7.55 (1H, td), 7.49-7.44 (2H, m), 3.63 (3H, s).

### e) 4′-Chloro-3′-[[(cyclohexylmethyl)amino]carbonyl]-[1,1′-biphenyl]-2-carboxylic acid, methyl ester

N,N-Dimethylformamide (1 drop) and oxalyl chloride (0.16 mL) were added to a stirred solution of 4′-chloro-[1,1′-biphenyl]-2,3′-dicarboxylic acid, 2-methyl ester (Example 3 (d)) (170 mg) in dichloromethane (2 mL) at 0° C. The reaction was allowed to warm to room temperature, stirred under nitrogen for 2 hours, then evaporated to dryness. The residue was dissolved in dichloromethane (2 mL) and cooled to 0° C. Cyclohexylmethylamine (80 mg) was added followed by triethylamine (0.16 mL). The reaction was allowed to warm to room temperature and stirred under nitrogen for 2 hours then poured into saturated aqueous sodium bicarbonate (20 mL). The aqueous was extracted with dichloromethane (3×20 mL). The combined organics were dried, filtered and evaporated. Purification (SiO₂, 1:3 ethyl acetate:isohexane) afforded the sub-title compound as a solid (190 mg).

¹H NMR (400 MHz, d₆-DMSO) δ 8.45 (1H, t), 7.79 (1H, dd), 7.66 (1H, td), 7.56-7.50 (2H, m), 7.47 (1H, dd), 7.34 (1H, dd), 7.29 (1H, d), 3.63 (3H, s), 3.08 (2H, t), 1.79-1.45 (6H, m), 1.29-1.07 (3H, m), 1.02-0.07 (2H, m).

### f) 4′-Chloro-3′-[[(cyclohexylmethyl)amino]carbonyl]-[1,1′-biphenyl]-2-carboxylic acid

A solution of potassium hydroxide (100 mg) in water (1 mL) was added to a solution of 4′-chloro-3′-[[(cyclohexylmethyl)amino]carbonyl]-[1,1′-biphenyl]-2-carboxylic acid, methyl ester (Example 3 (e)) (190 mg), in methanol (1 mL) and tetrahydrofuran (1 mL). The mixture was stirred at room temperature for 2 hours then concentrated. The residue was dissolved in water (5 mL) and the solution was acidified to pH 2 with 2M aqueous hydrochloric acid. The resulting solid was collected by filtration and washed with water (10 mL) to afford the title compound as a solid (150 mg).

MS: APCI(−ve) 370 (M−H⁺).

m.p. 212-214° C.

¹H NMR (400 MHz, d₆-DMSO) δ 12.92 (1H, s), 8.45 (1H, t), 7.79 (1H, dd), 7.61 (1H, td), 7.53-7.47 (2H, m), 7.41 (1H, dd), 7.37 (1H, dd), 7.33 (1H, d), 3.07 (2H, t), 1.79-1.45 (6H, m), 1.26-1.07 (3H, m), 0.99-0.86 (2H, m).

### EXAMPLES 4-9

The following examples were prepared by the general procedure of Example 3 (e)/(f) using 4′-chloro-[1,1′-biphenyl]-2,3′-dicarboxylic acid, 2-methyl ester (Example 3 (d)) and the appropriate amine.

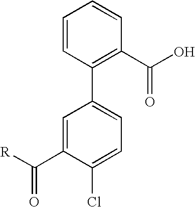

| Ex | Compound | R | Data |
|---|---|---|---|
| 4 | 4′-Chloro-3′-[[[(2S)-2-phenylpropyl]amino]carbonyl]-[1,1′-biphenyl]-2-carboxylic acid | 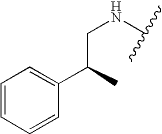 | ¹H NMR (400 MHz, d₆-DMSO) δ12.92 (1H, s), 8.50 (1H, t), 7.79(1H, dd), 7.61 (1H, td), 7.53-7.46(2H, m), 7.38-7.33 (2H, m), 7.32-7.24 (4H, m), 7.21-7.15 (2H, m),3.44-3.33 (2H, m), 3.04 (1H,sextet), 1.25 (3H,d).MS: APCI(−ve) 392 (M − H⁺).m.p. 160-162° C. |
| 5 | 4′-Chloro-3′-[[[[(1S,2R,5S)-6,6-dimethylbicyclo[3.1.1]hept-2-yl]methyl]amino]car-bonyl]-[1,1′-biphenyl]-2-carboxylic acid | 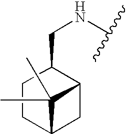 | ¹H NMR (400 MHz, d₆-DMSO) δ8.45 (1H, t), 7.76 (1H, dd), 7.59(1H, td), 7.53-7.45 (2H, m), 7.41-7.35 (2H, m), 7.32 (1H, d), 3.24(2H, dd), 2.38-2.20 (2H, m), 2.01-1.76 (5H, m), 1.58-1.45 (1H, m),1.17 (3H, s), 1.05 (3H, s), 0.86(1H, d).MS: APCI(+ve) 412 (M + H⁺).m.p. 170-172° C. |
| 6 | 4′-Chloro-3′-[[(cycloheptylmethyl)amino]carbonyl]-[1,1′-biphenyl]-2-carboxylic acid | 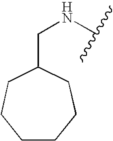 | ¹H NMR (400 MHz, d₆-DMSO) δ12.93 (1H, s), 8.48 (1H, t), 7.78(1H, dd), 7.61 (1H, td), 7.53-7.47(2H, m), 7.40 (1H, dd), 7.37 (1H,dd), 7.32 (1H, d), 3.06 (2H, t),1.78-1.31 (11H, m), 1.25-1.12(2H, m).MS:APCI (−ve) 384/386 (M − H⁺).m.p. 201-203° C. |
| 7 | 4′-Chloro-3′-[[[(1-hydroxycyclohexyl)methyl]amino]carbonyl]-[1,1′-biphenyl]-2-carboxylic acid | 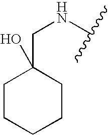 | ¹H NMR (400 MHz, d₆-DMSO) δ12.92 (1H, s), 8.24 (1H, t), 7.79(1H, dd), 7.62 (1H, td), 7.56-7.46(2H, m), 7.46-7.31 (3H, m), 4.23(1H, s), 3.23 (2H, d), 1.63-1.09(10H, m).MS: APCI(−ve) 386 (M − H⁺).m.p. 202-204° C. |
| 8 | 4′-Chloro-3′-[[[[cis-2-hydroxycycloheptyl]methyl]amino]carbonyl]-[1,1′-biphenyl]-2-carboxylic acid | 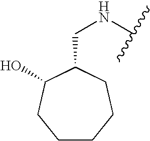 | ¹H NMR (400 MHz, d₆-DMSO) δ12.92 (1H, s), 8.41 (1H, s), 7.79(1H, d), 7.61 (1H, td), 7.55-7.46(2H, m), 7.45-7.31 (3H, m), 3.92(1H, d), 3.32-3.09 (2H, m), 1.83-1.15 (11H, m).MS: APCI(+ve) 402 (M + H⁺).m.p. 193-195° C. |
| 9 | 4′-Chloro-3′-[[(2-cyclohexylethyl)amino]carbonyl]-[1,1′-biphenyl]-2-carboxylic acid | 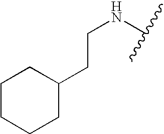 | ¹H NMR (400 MHz, d₆-DMSO) δ12.91 (1H, s), 8.41 (1H, t), 7.79(1H, d), 7.61 (1H, ddd), 7.53-7.47 (2H, m), 7.42-7.35 (2H, m),7.32 (1H, d), 3.25 (2H, q), 1.77-1.56 (5H, m), 1.45-1.29 (3H, m),1.26-1.06 (3H, m), 0.97-0.83(2H,m).MS: APCI(+ve) 386 (M + H⁺).m.p. 191-193° C. |

### EXAMPLE 10

### 3-[4-Chloro-3-[[(cycloheptylmethyl)amino]carbonyl]phenyl]-2-pyridinecarboxylic acid

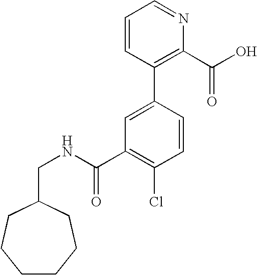

### a) 3-Iodo-2-pyridinecarboxylic acid, methyl ester

Butyllithium (32 mL, 2.5 M in hexanes) was added dropwise over 10 minutes to a solution of 2,2,6,6-tetramethylpiperidine (10.2 mL) in tetrahydrofuran (100 mL) at −78° C. under nitrogen. The mixture was stirred at −78° C. for 15 minutes and then picolinic acid (2.4 g) was added portionwise over 10 minutes. After a further 10 minutes at −78° C. the mixture was allowed to warm to 0° C. and stirred under nitrogen for 30 minutes. The reaction mixture was then added dropwise over 15 minutes to a solution of iodine (15 g) in tetrahydrofuran (100 mL) at 0° C. This was then allowed to warm to room temperature and stirred for 1 hour before water (20 mL) was added. The mixture was evaporated to dryness to leave a black oil. Dichloromethane (50 mL) was added and the mixture was cooled to 0° C. N,N-Dimethylformamide (1 drop) and oxalyl chloride (4 mL) were added. The reaction was allowed to warm to room temperature and stirred under nitrogen for 2 hours, then evaporated to dryness. The residue was dissolved in dichloromethane (20 mL) and then methanol (20 mL) was added. The mixture was then stirred for 10 minutes before being evaporated to afford the sub-title compound as an oil (1.0 g) which was used in the next step without purification.

MS: APCI(+ve) 264 (M+H⁺).

### b) 3-[4-Chloro-3-[(1,1-dimethylethoxy)carbonyl]phenyl]-2-pyridinecarboxylic acid, methyl ester

2-Chloro-5-(4,4,5,5-tetramethyl-1,3,2-dioxaborolan-2-yl)-benzoic acid, 1,1-dimethylethyl ester (Example 3 (b)) (500 mg), 3-iodo-2-pyridinecarboxylic acid, methyl ester (Example 10 (a)) (400 mg) and tetrahydrofuran (2 mL) were placed in a 10 mL microwave vial. A solution of potassium carbonate (400 mg) in water (1 mL) was added followed by bis(triphenylphosphine)palladium(II) chloride (50 mg), and the mixture was heated to 130° C. in a microwave for 3 hours then concentrated. The residue was partitioned between dichloromethane (20 n3L) and water (20 mL). The layers were separated and the aqueous was extracted with dichloromethane (2×20 mL). The combined organics were dried, filtered and evaporated. Purification by chromatography (SiO₂, 1:4 ethyl acetate:isohexane as eluant) gave the sub-title compound as a solid (240 mg).

MS: APCI(+ve) 348/450 (M+H⁺).

¹H NMR (400 MHz, d₆-DMSO) δ 8.68 (1H, dd), 8.02 (1H, dd), 7.71-7.63 (3H, m), 7.54 (1H, dd), 3.71 (3H, s), 1.56 (9H, s).

### c) 3-(3-Carboxy-4-chlorophenyl)-2-pyridinecarboxylic acid, 2-methyl ester

Prepared according to the method of Example 3 (d), using 3-[4-chloro-3-[(1,1-dimethylethoxy)carbonyl]phenyl]-2-pyridinecarboxylic acid, methyl ester (Example 10 (b)) (240 mg), trifluoroacetic acid (1 mL) and dichloromethane (3 mL) to afford the sub-title compound as an oil (200 mg).

MS: APCI(+ve) 292/294 (M+H⁺).

¹H NMR (300 MHz, CDCl₃) δ 8.97 (1H, dd), 8.14 (1H, dd), 8.00 (1H, d), 7.91 (1H, dd), 7.64 (1H, d), 7.48 (1H, dd), 3.88 (3H, d).

### d) 3-[4-Chloro-3-[[(cycloheptylmethyl)amino]carbonyl]phenyl]-2-pyridinecarboxylic acid, methyl ester

Prepared according to the method of Example 3 (e), using 3-(3-carboxy-4-chlorophenyl)-2-pyridinecarboxylic acid, 2-methyl ester (Example 10 (c)) (170 mg), N,N-dimethylformamide (1 drop), oxalyl chloride (1 mL), (cycloheptylmethyl)amine (90 mg), triethylamine (0.2 mL) and dichloromethane (4 mL). Purification by chromatography (SiO₂, 2:3 ethyl acetate:isohexane) afforded the sub-title compound as a solid (170 mg).

MS: APCI(+ve) 401/403 (M+H⁺).

¹H NMR (400 MHz, CDCl₃) δ 8.72 (1H, d), 7.75 (1H, d), 7.67 (1H, d), 7.51 (1H, dd), 7.46 (1H, d), 7.32 (1H, dd), 6.32 (1H, s), 3.85 (3H, s), 3.34 (2H, t), 1.86-1.40 (11H, m), 1.35-1.20 (2H, m).

### e) 3-[4-Chloro-3-[[(cycloheptylmethyl)amino]carbonyl]phenyl]-2-pyridinecarboxylic acid

Prepared according to the method of Example 3 (f), using 3-[4-chloro-3-[[(cycloheptylmethyl)amino]carbonyl]phenyl]-2-pyridinecarboxylic acid, methyl ester (Example 10 (d)) (175 mg), potassium hydroxide (100 mg), water (1 mL), methanol (1 mL) and tetrahydrofuran (1 mL) to afford the title compound as a solid (150 mg).

MS: APCI(−ve) 385 (M−H⁺).

m.p. 166-168° C.

¹H NMR (400 MHz, d₆-DMSO) δ 13.45 (1H, s), 8.64 (1H, dd), 8.51 (1H, t), 7.93 (1, dd), 7.63 (1H, dd), 7.58 (1H, d), 7.47 (1H, dd), 7.44 (1H, d), 3.07 (2H, t), 1.80-1.33 (11H, m), 1.25-1.13 (2H, m).

### EXAMPLE 11

### 3-[4-Chloro-3-[[(2-cyclohexylethyl)amino]carbonyl]phenyl]-2-pyridinecarboxylic acid

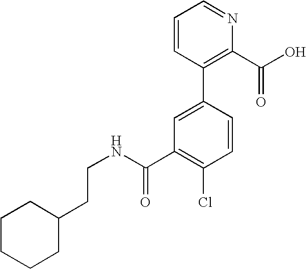

### a) 3-[4-Chloro-3-[[(2-cyclohexylethyl)amino]carbonyl]phenyl]-2-pyridinecarboxylic acid, methyl ester

Prepared according to the method of Example 3 (e), using 3-(3-carboxy-4-chlorophenyl)-2-pyridinecarboxylic acid, 2-methyl ester (Example 10 (c)) (170 mg), N,N-dimethylformamide (1 drop), oxalyl chloride (1 mL), (2-cyclohexylethyl)amine hydrochloride (90 mg), triethylamine (0.25 mL) and dichloromethane (4 mL). Purification by chromatography (SiO₂, 2:3 ethyl acetate:isohexane) afforded the sub-title compound as a solid (170 mg).

MS: APCI(+ve) 401/403 (M+H⁺).

¹H NMR (400 MHz, CDCl₃) δ 8.71 (1H, dd), 7.74 (1H, dd), 7.66 (1H, d), 7.51 (1H, dd), 7.45 (1H, d), 7.32 (1H, dd), 6.23 (1H, s), 3.85 (3H, s), 3.54-3.47 (2H, m), 1.84-1.48 (7H, m), 1.45-1.33 (1H, m), 1.32-1.08 (3H, m), 1.04-0.88 (2H, m).

### b) 3-[4-Chloro-3-[[(2-cyclohexylethyl)amino]carbonyl]phenyl]-2-pyridinecarboxylic acid

Prepared according to the method of Example 3 (f), using 3-[4-chloro-3-[[(2-cyclohexylethyl)amino]carbonyl]phenyl]-2-pyridinecarboxylic acid, methyl ester (Example 11 (a)) (175 mg), potassium hydroxide (100 mg), water (1 mL), methanol (1 mL) and tetrahydrofuran (1 mL) to afford the title compound as a solid (125 mg).

MS: APCI(−ve) 385 (M−H⁺).

m.p. 104-107° C.

¹H NMR (400 MHz, d₆-DMSO) δ 8.64 (1H, dd), 8.44 (1H, t), 7.93 (1H, dd), 7.63 (1H, dd), 7.58 (1H, d), 7.47 (1H, dd), 7.44 (1H, d), 3.25 (2H, q), 1.77-1.56 (5H, m), 1.45-1.29 (3H, m), 1.25-1.06 (3H, m), 0.96-0.81 (2H, m).

### EXAMPLE 12

### 4′-Chloro-3′-[[[(1R)-1-cyclohexylethyl]amino]carbonyl]-[1,1′-biphenyl]-2-carboxylic acid

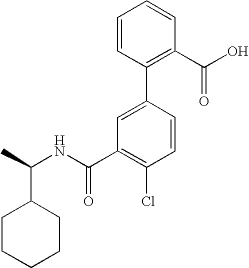

### a) 4′-Chloro-3′-[[[(1R)-1-cyclohexylethyl]amino]carbonyl]-[1,1′-biphenyl]-2-carboxylic acid, methyl ester

Prepared according to the method of Example 3 (e), using 4′-chloro-[1,1′-biphenyl]-2,3′-dicarboxylic acid, 2-methyl ester (Example 3 (d)) (170 mg), N,N-dimethylformamide (1 drop), oxalyl chloride (0.16 mL), (αR)-α-methylcyclohexanemethanamine (90 mg), triethylamine (0.16 mL) and dichloromethane (4 mL). Purification by chromatography (SiO₂, 1:4 ethyl acetate:isohexane) afforded the sub-title compound as a colourless oil (190 mg).

MS: APCI(+ve) 400/402 (M+H⁺).

¹H NMR (400 MHz, CDCl₃) δ 7.89 (1H, dd), 7.61 (1H, d), 7.55 (1H, td), 7.44 (1H, td), 7.41 (1H, d), 7.35 (1H, dd), 7.28 (1H, dd), 6.05 (1H, d), 4.18-4.04 (1H, m), 3.72 (3H, s), 1.91-1.63 (5H, m), 1.50-1.38 (1H, m), 1.30-1.00 (8H, m).

### b) 4′-Chloro-3′-[[[(1R)-1-cyclohexylethyl]amino]carbonyl]-[1,1′-biphenyl]-2-carboxylic acid

Prepared according to the method of Example 3 (f), using 4′-chloro-3′-[[[(1R)-1-cyclohexylethyl]amino]carbonyl]-[1,1′-biphenyl]-2-carboxylic acid, methyl ester (Example 12 (a)) (190 mg), potassium hydroxide (100 mg), water (1 mL), methanol (1 mL) and tetrahydrofuran (1 mL) to afford the title compound as a solid (160 mg).

MS: APCI(+ve) 386 (M+H⁺).

m.p. 139-141° C.

¹H NMR (300 MHz, d₆-DMSO) δ 12.92 (1H, s), 8.27 (1H, d), 7.79 (1H, d), 7.61 (1H, t), 7.54-7.46 (2H, m), 7.43-7.33 (2H, m), 7.29 (1H, s), 3.88-3.71 (1H, m), 1.87-1.52 (5H, m), 1.45-1.29 (1H, m), 1.28-0.88 (8H, m).

### EXAMPLE 13

### 4′-Chloro-3′-[[[(1-methylcycloheptyl)methyl]amino]carbonyl]-[1,1′-biphenyl]-2-carboxylic acid

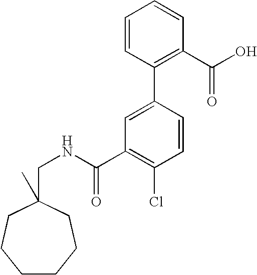

### a) 1-Methyl-cycloheptanecarbonitrile

A solution of cycloheptanecarbonitrile (500 mg) in tetrahydrofuran (1 mL) was added dropwise to lithium diisopropylamide (2.8 mL, 1.8 M solution in tetrahydrofuran) at −40° C. under an atmosphere of nitrogen. The mixture was allowed to warm to −20° C. for 10 minutes, then cooled to −40° C. again. Methyl iodide (0.35 mL) was added dropwise and the reaction was allowed to warm to room temperature and stirred for 1 hour. The reaction mixture was concentrated and the residue partitioned between diethyl ether (20 mL) and 2M aqueous hydrochloric acid (20 mL). The layers were separated and the organic layer was dried, filtered and evaporated to afford the sub-title compound as a yellow oil (600 mg).

¹H NMR (400 MHz, CDCl₃) δ 2.04-1.95 (2H, m), 1.75-1.61 (6H, m), 1.59-1.45 (4H, m), 1.36 (3H, s).

### b) 1-Methyl-cycloheptanemethanamine

A solution of 1-methyl-cycloheptanecarbonitrile (Example 13 (a)) (550 mg) in tetrahydrofuran (8 mL) was added dropwise to lithium aluminium hydride (12 mL, 1M solution in tetrahydrofuran) at room temperature under an atmosphere of nitrogen. The mixture was heated to 50° C. for 3 hours then cooled to 0° C. and quenched by careful addition of water (1 mL), followed by 15% aqueous sodium hydroxide (1 mL) then water (2 mL). The mixture was filtered through diatomaceous earth and then partitioned between water (50 mL) and diethyl ether (50 mL). The layers were separated and the organic layer dried, filtered and evaporated to afford the sub-title compound as a yellow oil (500 mg).

¹H NMR (400 MHz, CDCl₃) δ 2.42 (2H, s), 1.58-1.24 (12H, m), 0.83 (3H, s).

### c) 4′-Chloro-3′-[[[(1-methylcycloheptyl)methyl]amino]carbonyl]-[1,1′-biphenyl]-2-carboxylic acid, methyl ester

Prepared according to the method of Example 3 (e), using 4′-chloro-[1,1′-biphenyl]-2,3′-dicarboxylic acid, 2-methyl ester (Example 3 (d)) (170 mg), N,N-dimethylformamide (1 drop), oxalyl chloride (0.16 mL), 1-methyl-cycloheptanemethanamine (Example 13 (b)) (165 mg), triethylamine (0.17 mL) and dichloromethane (4 mL). Purification by chromatography (SiO₂, 1:9 ethyl acetate:isohexane) afforded the sub-title compound as a solid (180 mg).

MS: APCI(+ve) 414/416 (M+H⁺).

¹H NMR (300 MHz, CDCl₃) δ 7.90 (1H, d), 7.65 (1H, d), 7.55 (1H, td), 7.44 (1H, td), 7.42 (1H, d), 7.35 (1H, d), 7.28 (1H, dd), 6.31 (1H, s), 3.72 (3H, s), 3.31 (2H, d), 1.65-1.33 (12H, m), 0.97 (3H, s).

### d) 4′-Chloro-3′-[[[(1-methylcycloheptyl)methyl]amino]carbonyl]-[1,1′-biphenyl]-2-carboxylic acid

Prepared according to the method of Example 3 (f), using 4′-chloro-3′-[[[(1-methylcycloheptyl)methyl]amino]carbonyl]-[1,1′-biphenyl]-2-carboxylic acid, methyl ester (Example 13 (c)) (180 mg), potassium hydroxide (100 mg), water (1 mL), methanol (1 mL) and tetrahydrofuran (1 mL). The reaction mixture was concentrated, the residue was dissolved in water (5 mL) and the solution was acidified to pH 2 with 2M aqueous hydrochloric acid. This was extracted with dichloromethane (3×10 mL), the combined extracts were dried, filtered and evaporated and the resulting solid was recrystallised from acetonitrile to afford the title compound as a solid (160 mg).

MS: APCI(+ve) 400 (M+H⁺).

m.p. 180-183° C.

¹H NMR (300 MHz, d₆-DMSO) δ 12.93 (1H, s), 8.40 (1H, t), 7.78 (1H, d), 7.61 (1H, t), 7.56-7.46 (2H, m), 7.44-7.30 (3H, m), 3.08 (2H, d), 1.59-1.18 (12H, m), 0.88 (3H, s).

### EXAMPLE 14

### 4′-Chloro-3′-[[[[1-(hydroxymethyl)cycloheptyl]methyl]amino]carbonyl]-[1,1′-biphenyl]-2-carboxylic acid

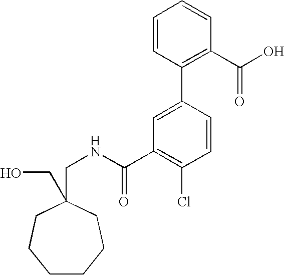

### a) 1-Cyano-cycloheptanecarboxylic acid, ethyl ester

A solution of cycloheptanecarbonitrile (200 mg) in tetrahydrofuran (0.5 mL) was added dropwise to lithium diisopropylamide (1.1 mL, 1.8 M solution in tetrahydrofuran) at −40° C. under an atmosphere of nitrogen. The mixture was allowed to warm to −20° C. for 10 minutes, and then cooled to −40° C. again. A solution of ethyl chloroformate (0.23 mL) in tetrahydrofuran (0.5 mL) was added dropwise and the reaction was allowed to warm to room temperature and stirred overnight. The reaction mixture was concentrated and the residue partitioned between diethyl ether (20 mL) and 2M aqueous hydrochloric acid (20 mL). The layers were separated and the organic layer was dried, filtered and evaporated. Purification (SiO₂, 1:49 ethyl acetate: isohexane) afforded the sub-title compound as a colourless oil (110 mg).

¹H NMR (400 MHz, CDCl₃) δ 4.25 (2H, q), 2.21-2.12 (2H, m), 2.09-2.00 (2H, m), 1.83-1.65 (6H, m), 1.63-1.53 (2H, m), 1.32 (3H, t).

### b) 1-(Aminomethyl)-cycloheptanemethanol

Prepared according to the method of Example 13 (b), using 1-cyano-cycloheptanecarboxylic acid, ethyl ester (Example 15 (a)) (110 mg), lithium aluminium hydride (2.8 mL, 1 M solution in tetrahydrofuran) and tetrahydrofuran (0.5 mL) to afford the sub-title compound as a white solid (55 mg).

¹H NMR (400 MHz, CDCl₃) δ 3.51 (2H, s), 2.76 (2H, s), 1.60-1.37 (10H, m), 1.35-1.24 (2H, m).

### c) 4′-Chloro-3′-[[[[1-(hydroxymethyl)cycloheptyl]methyl]amino]carbonyl]-[1,1′-biphenyl]-2-carboxylic acid, methyl ester

Prepared according to the method of Example 3 (e), using 4′-chloro-[1,1′-biphenyl]-2,3′-dicarboxylic acid, 2-methyl ester (Example 3 (d)) (100 mg), N,N-dimethylformamide (1 drop), oxalyl chloride (0.16 mL), 1-(aminomethyl)-cycloheptanemethanol (Example 14 (b)) (55 mg), triethylamine (0.1 mL) and dichloromethane (2 mL). Purification by chromatography (SiO₂, 1:4 ethyl acetate:isohexane) afforded the sub-title compound as a solid (80 mg).

MS: APCI(+ve) 430 (M+H⁺).

### d) 4′-Chloro-3′-[[[[1-(hydroxymethyl)cycloheptyl]methyl]amino]carbonyl]-[1,1′-biphenyl]-2-carboxylic acid

Prepared according to the method of Example 3 (f), using 4′-chloro-3′-[[[[1-(hydroxymethyl)cycloheptyl]methyl]amino]carbonyl]-[1,1′-biphenyl]-2-carboxylic acid, methyl ester (Example 14 (c)) (80 mg), potassium hydroxide (100 mg), water (1 mL), methanol (1 mL) and tetrahydrofuran (1 mL). The reaction mixture was concentrated, the residue was dissolved in water (5 mL) and the solution was acidified to pH 2 with 2M aqueous hydrochloric acid. This was extracted with dichloromethane (3×10 mL), the combined extracts were dried, filtered and evaporated and the resulting solid was recrystallised from acetonitrile to afford the title compound as a solid (45 mg).

MS: APCI(−ve) 414 (M−H⁺).

m.p. 175-177° C.

¹H NMR (300 MHz, d₆-DMSO) δ 8.41 (1H, t), 7.79 (1H, dd), 7.61 (1H, td), 7.53 (1H, d), 7.50 (1H, td), 7.44-7.34 (3H, m), 3.18-3.08 (4H, m), 1.57-1.21 (12H, m).

### EXAMPLE 15

### 3-[4-Chloro-3-[[[(1-hydroxycycloheptyl)methyl]amino]carbonyl]phenyl]-2-pyridinecarboxylic acid

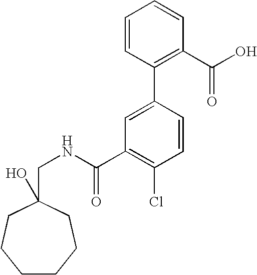

### a) 3-[4-Chloro-3-[[[(1-hydroxycycloheptyl)methyl]amino]carbonyl]phenyl]-2-pyridinecarboxylic acid, methyl ester

Prepared according to the method of Example 3 (e), using 3-(3-carboxy-4-chlorophenyl)-2-pyridinecarboxylic acid, 2-methyl ester (Example 10 (c)) (170 mg), N,N-dimethylformamide (1 drop), oxalyl chloride (1 mL), 1-(aminomethyl)-cycloheptanol (Example 2 (b)) (250 mg), triethylamine (0.2 mL) and dichloromethane (4 mL). Purification by chromatography (SiO₂, 1:30 methanol:dichloromethane) afforded the sub-title compound as a solid (200 mg).

MS: APCI(−ve) 415/417 (M−H⁺).

¹H NMR (400 MHz, CDCl₃) δ 8.72 (1H, dd), 7.75 (1H, dd), 7.67 (1H, d), 7.51 (1H, dd), 7.47 (1H, d), 7.33 (1H, dd), 6.78-6.68 (1H, m), 3.84 (3H, s), 3.49 (2H, d), 1.78-1.42 (12H, m).

### b) 3-[4-Chloro-3-[[[(1-hydroxycycloheptyl)methyl]amino]carbonyl]phenyl]-2-pyridinecarboxylic acid

Prepared according to the method of Example 3 (f), using 3-[4-chloro-3-[[[(1-hydroxycycloheptyl)methyl]amino]carbonyl]phenyl]-2-pyridinecarboxylic acid, methyl ester (Example 15 (a)) (200 mg), potassium hydroxide (100 mg), water (1 mL), methanol (1 mL) and tetrahydrofuran (1 mL). Purification by RP-HPLC (acetonitrile:aqueous trifluoroacetic acid, Symmetry) gave the title compound as a solid (45 mg).

MS: APCI(−ve) 401 (M−H⁺).

m.p. 95-100° C.

¹H NMR (400 MHz, d₆-DMSO) δ 8.64 (1H, dd), 8.26 (1H, t), 7.95 (1H, dd), 7.64 (1H, dd), 7.58 (1H, d), 7.51 (1H, d), 7.47 (1H, dd), 3.23 (2H, d), 1.68-1.30 (12H, m).

### EXAMPLE 16

### 3′-[[(Cycloheptylmethyl)amino]carbonyl]-4′-methyl-[1,1′-biphenyl]-2-carboxylic acid

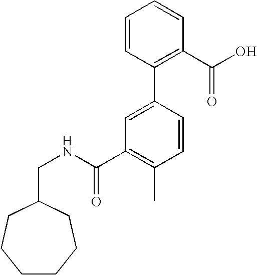

### a) 5-Bromo-N-(cycloheptylmethyl)-2-methyl-benzamide

To a stirred solution of 5-bromo-2-methyl-benzoic acid (U.S. Pat. No. 4,282,365) (1 g) in dichloromethane (20 mL) was added N,N-dimethylformamide (1 drop) followed by oxalyl chloride (1.6 mL). The reaction was stirred for two hours, the volatiles were removed under vacuum and dichloromethane (20 mL), cycloheptanemethanamine (649 mg) and triethylamine (1.29 mL) were added. The reaction was stirred for 30 minutes before the reaction was acidified with 2M hydrochloric acid. The aqueous phase was separated, the organic phase was washed once with brine, dried over magnesium sulphate, filtered and the solvent removed to afford the sub-title compound (1.48 g).

MS: APCI(+ve) 324 (M+H⁺).

¹H NMR (300 MHz, d₆-DMSO) δ 8.43-8.33 (1H, m), 7.50 (1H, dd), 7.45-7.40 (1H, m), 7.25-7.18 (1H, m), 3.10-3.02 (2H, m), 2.30-2.24 (3H, m), 1.79-1.32 (11H, m), 1.31-1.11 (2H, m).

### b) N-(Cycloheptylmethyl)-2-methyl-5-(4,4,5,5-tetramethyl-1,3,2-dioxaborolan-2-yl)-benzamide

5-Bromo-N-(cycloheptylmethyl)-2-methyl-benzamide (Example 16 (a)) (1.48 g), tetrakis(triphenylphosphine)palladium (15 mg), potassium acetate (2 g), 4,4,4′,4′,5,5,5′,5′-octamethyl-2,2′-bi-1,3,2-dioxaborolane (1.85 g) in N,N-dimethylformamide (15 mL) were heated under nitrogen for 3 hours at 90° C. with stirring. Tetrakis(triphenylphosphine)palladium (130 mg), potassium acetate (200 mg) and 4,4,4′,4′,5,5,5′,5′-octamethyl-2,2′-bi-1,3,2-dioxaborolane (200 mg) was added and the mixture was heated at 90° C. for an additional 15 hours. The reaction was worked up by the addition of ethyl acetate/water, the organic phase was separated and the aqueous phase was further extracted twice with ethyl acetate. The combined organic fractions were washed once with water, once with brine, dried over magnesium sulphate, filtered and the solvent removed in vacuo. Purification by chromatography on SiO₂, eluting with dichloromethane, gave the sub-title compound (700 mg).

MS: APCI(+ve) 372 (M+H⁺).

### c) 3′-[[(Cycloheptylmethyl)amino]carbonyl]-4′-methyl-[1,1′-biphenyl]-2-carboxylic acid

N-(Cycloheptylmethyl)-2-methyl-5-(4,4,5,5-tetramethyl-1,3,2-dioxaborolan-2-yl)-benzamide (Example 16 (b)) (150 mg), 2-bromo-benzoic acid, methyl ester (130 mg), tetrakis(triphenylphosphine)palladium (15 mg), sodium carbonate (128 mg), tetrahydrofuran (2 mL) and water (1 mL) were heated in a microwave at 120° C. for 40 minutes. 48% w/v sodium hydroxide solution (0.3 mL) and methanol (1 mL) were added to the reaction and the mixture was heated at 90° C. for 30 minutes in a microwave. The products were acidified with acetic acid and purified by RP-HPLC (0.2% trifluoroacetic acid/acetonitrile, Xterra column). The solvent was removed in vacuo and the resulting solid was triturated with acetonitrile, filtered and dried under vacuum to afford the title compound (32 mg).

MS: APCI(+ve) 366 (M+H⁺).

m.p. 188-189° C.

¹H NMR (400 MHz, d₆-DMSO) δ 12.81 (1H, s), 8.28 (1H, t), 7.72 (1H, dd), 7.58 (1H, td), 7.45 (1H, td), 7.42 (1H, dd), 7.28-7.25 (3H, m), 3.06 (2H, t), 2.36 (3H, s), 1.76-1.33 (11H, m), 1.25-1.12 (2H, m).

### EXAMPLE 17

### 1-[3-[3-[[(Cycloheptylmethyl)amino]carbonyl]-4-methylphenyl]-2-pyridinyl]-4-piperidinecarboxylic acid

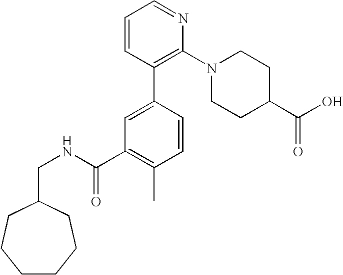

### a) 1-(3-Bromo-2-pyridinyl)-4-piperidinecarboxylic acid, methyl ester

A mixture of 2,3-dibromo-pyridine (3.0 g) and 4-piperidinecarboxylic acid, methyl ester (5.4 g) was heated at 130° C. in a microwave for 30 minutes. The products were concentrated in vacuo and purified by chromatography (SiO₂, dichloromethane as eluant) to give the sub-title compound (2.4 g) as a colourless oil.

¹H NMR (400 MHz, d₆-DMSO) δ 8.24 (1H, dd), 7.95 (1H, dd), 6.92 (1H, dd), 3.63 (3H, s), 3.62-3.59 (2H, m), 2.88-2.79 (2H, m), 2.60-2.52 (1H, m), 1.97-1.89 (2H, m), 1.78-1.66 (2H, m).

### b) 1-[3-[3-[[(Cycloheptylmethyl)amino]carbonyl]-4-methylphenyl]-2-pyridinyl]-4-piperidinecarboxylic acid

Prepared according to the method of Example 16 (c) using N-(cycloheptylmethyl)-2-methyl-5-(4,4,5,5-tetramethyl-1,3,2-dioxaborolan-2-yl)-benzamide (Example 16 (b)) (150 mg) and 1-(3-bromo-2-pyridinyl)-4-piperidinecarboxylic acid, methyl ester (133 mg) to afford the title compound (114 mg).

MS: APCI(−ve) 448 (M−H⁺).

¹HNMR (400 MHz, d₆-DMSO) δ 8.26 (1H, t), 8.18 (1H, dd), 7.72 (1H, d), 7.58 (1H, dd), 7.53 (1H, d), 7.33 (1H, d), 7.09 (1H, dd), 3.46-3.39 (2H, m), 3.07 (2H, t), 2.79-2.70 (2H, m), 2.37 (3H, s), 2.36-2.29 (1H, m), 1.77-1.33 (15H, m), 1.25-1.13 (2H, m).

### EXAMPLE 18

### 3-Chloro-6-[3-[[(cycloheptylmethyl)amino]carbonyl]-4-methylphenyl]-2-pyridinecarboxylic acid

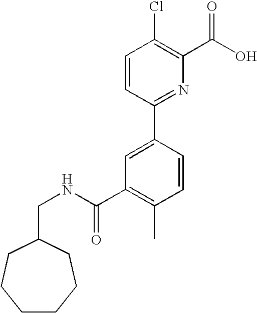

Prepared according to the method of Example 16 (c) using N-(cycloheptylmethyl)-2-methyl-5-(4,4,5,5-tetramethyl-1,3,2-dioxaborolan-2-yl)-benzamide (Example 16 (b)) (150 mg) and 3,6-dichloro-2-pyridinecarboxylic acid, methyl ester (83 mg) to afford the title compound (22 mg).

MS: APCI(−ve) 399 (M−H⁺).

m.p. 177-178° C.

¹H NMR (400 MHz, d₆-DMSO) δ 13.90 (1H, s), 8.38 (1H, t), 8.13 (2H, s), 8.03 (1H, dd), 8.00 (1H, d), 7.38 (1H, d), 3.10 (2H, t), 2.37 (3H, s), 1.78-1.35 (11H, m), 1.26-1.15 (2H, m).

### EXAMPLE 19

### 5-Chloro-2-[4-chloro-3-[[(2-cyclohexylethyl)amino]carbonyl]phenyl]-3-pyridinecarboxylic acid

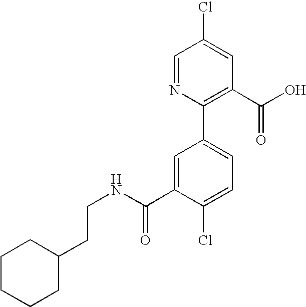

### a) 5-Chloro-2-[4-chloro-3-[(1,1-dimethylethoxy)carbonyl]phenyl]-3-pyridinecarboxylic acid, methyl ester

Prepared according to the method of Example 3 (c) using 2-chloro-5-(4,4,5,5-tetramethyl-1,3,2-dioxaborolan-2-yl)-benzoic acid, 1,1-dimethylethyl ester (Example 3 (b)) (0.8 g) and 2,5-dichloro-3-pyridinecarboxylic acid, methyl ester (0.49 g), stirring at 65° C. under nitrogen for 2 hours. The products were filtered through diatomaceous earth, washing with methanol (2×20 mL) and concentrated in vacuo. The residue was partitioned between dichloromethane (50 mL) and water (25 mL), the layers were separated and the organic fraction was dried (MgSO₄), filtered and concentrated in vacuo. Purification by chromatography (SiO₂, 98:2 dichloromethane:methanol as eluant) gave the sub-title compound as a colourless oil (0.59 g).

¹H NMR (400 MHz, CDCl₃) δ 8.73 (1H, d), 8.13 (1H, d), 7.90 (1H, d), 7.55 (1H, dd), 7.49 (1H, d), 3.76 (3H, s), 1.61 (9H, s).

### b) 2-(3-Carboxy-4-chlorophenyl)-5-chloro-3-pyridinecarboxylic acid, 3-methyl ester

Prepared according to the method of Example 3 (d) using 5-chloro-2-[4-chloro-3-[(1,1-dimethylethoxy)carbonyl]phenyl]-3-pyridinecarboxylic acid, methyl ester (Example 19 (a)) (0.59 g) to give the sub-title compound as an oil (0.50 g).

MS: APCI(+ve) 326/328 (M+H⁺).

### c) 5-Chloro-2-[4-chloro-3-[[(2-cyclohexylethyl)amino]carbonyl]phenyl]-3-pyridinecarboxylic acid, methyl ester

To a solution of 2-(3-carboxy-4-chlorophenyl)-5-chloro-3-pyridinecarboxylic acid, 3-methyl ester (Example 19 (b)) (165 mg) in dichloromethane (5 mL) was added triethylamine (0.40 mL) followed by 2-(1H-benzotriazole-1-yl)-1,1,3,3-tetramethyluronium tetrafluoroborate (325 mg) and cyclohexaneethanamine hydrochloride (159 mg). The mixture was stirred at room temperature for 24 hours, dichloromethane (25 mL) and 2M aqueous hydrochloric acid (10 mL) were added and the layers were separated. The organic fraction was washed with saturated aqueous sodium hydrogen carbonate (10 mL), dried (MgSO₄), filtered and concentrated in vacuo before being purified by chromatography (SiO₂, dichloromethane, then 99:1 dichloromethane:methanol as eluant) to give the sub-title compound as a film (180 mg).

MS: APCI(+ve) 435/437 (M+H⁺).

### d) 5-Chloro-2-[4-chloro-3-[[(2-cyclohexylethyl)amino]carbonyl]phenyl]-3-pyridinecarboxylic acid

To a solution of 5-chloro-2-[4-chloro-3-[[(2-cyclohexylethyl)amino]carbonyl]phenyl]-3-pyridinecarboxylic acid, methyl ester (Example 19 (c)) (180 mg) in methanol (3 mL) was added a solution of sodium hydroxide (49 mg) in water (1 mL). The mixture was stirred at room temperature for 11 hours, concentrated in vacuo and 2M aqueous hydrochloric acid (5 mL) was added. The resulting precipitate was filtered, washed with water (5 mL) and recrystallised from acetonitrile to give the title compound as a solid (82 mg).

MS: APCI(−ve) 419/421 (M−H⁺).

m.p. 208-211° C.

¹H NMR (400 MHz, d₆-DMSO) δ 8.84 (1H, d), 8.47 (1H, t), 8.28 (1H, d), 7.58-7.54 (3H, m), 3.25 (2H, dt), 1.75-0.82 (13H, m).

### EXAMPLE 20

### 5-Chloro-2-[4-chloro-3-[[(cycloheptylmethyl)amino]carbonyl]phenyl]-3-pyridinecarboxylic acid

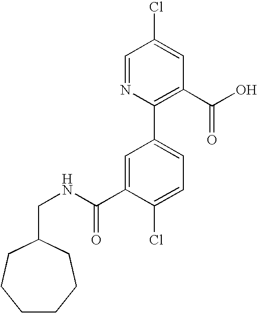

### a) 5-Chloro-2-[4-chloro-3-[[(cycloheptylmethyl)amino]carbonyl]phenyl]-3-pyridinecarboxylic acid, methyl ester

Prepared according to the method of Example 19 (c) using 2-(3-carboxy-4-chlorophenyl)-5-chloro-3-pyridinecarboxylic acids 3-methyl ester (Example 19 (b)) (165 mg) and cycloheptanemethanamine (129 mg) to give the sub-title compound as a film (180 mg).

MS: APCI(+ve) 435/437 (M+H⁺).

### b) 5-Chloro-2-[4-chloro-3-[[(cycloheptylmethyl)amino]carbonyl]phenyl]-3-pyridinecarboxylic acid

Prepared according to the method of Example 19 (d) using 5-chloro-2-[4-chloro-3-[[(cycloheptylmethyl)amino]carbonyl]phenyl]-3-pyridinecarboxylic acid, methyl ester (Example 20 (a)) (180 mg) to give the title compound as a solid (63 mg).

MS: APCI(−ve) 419/421 (M−H⁺).

m.p. 176-179° C.

¹H NMR (400 MHz, d₆-DMSO) δ 13.75 (1H, s), 8.85 (1H, d), 8.54 (1H, t), 8.28 (1H, d), 7.59-7.54 (3H, m), 3.07 (2H, t), 1.79-1.13 (13H, m).

### EXAMPLE 21

### 5-Chloro-2-[4-chloro-3-[[[(1-hydroxycycloheptyl)methyl]amino]carbonyl]phenyl]-3-pyridinecarboxylic acid

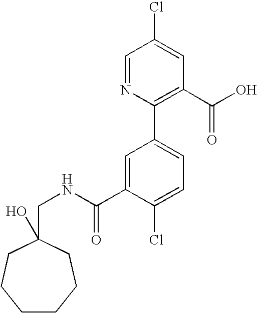

### a) 5-Chloro-2-[4-chloro-3-[[[(1-hydroxycycloheptyl)methyl]amino]carbonyl]phenyl]-3-pyridinecarboxylic acid, methyl ester

Prepared according to the method of Example 19 (c) using 2-(3-carboxy-4-chlorophenyl)-5-chloro-3-pyridinecarboxylic acid, 3-methyl ester (Example 19 (b)) (165 mg) and 1-(aminomethyl)-cycloheptanol (Example 2 (b)) (145 mg) to give the sub-title compound as a film (155 mg).

MS: APCI(+ve) 433/435 (M−H₂O+1H⁺).

### b) 5-Chloro-2-[4-chloro-3-[[[(1-hydroxycycloheptyl)methyl]amino]carbonyl]phenyl]-3-pyridinecarboxylic acid

Prepared according to the method of Example 19 (d) using 5-chloro-2-[4-chloro-3-[[[(1-hydroxycycloheptyl)methyl]amino]carbonyl]phenyl]-3-pyridinecarboxylic acid, methyl ester (Example 21 (a)) (155 mg) to give the title compound as a solid (10 mg).

MS: APCI(+ve) 437/439 (M+H⁺).

m.p. 201-204° C.

¹H NMR (400 MHz, d₆-DMSO) δ 8.50 (1H, s), 8.19-8.12 (1H, m), 7.88-7.80 (2H, m), 7.70 (1H, s), 7.46 (1H, d), 4.34 (1H, s), 3.23 (2H, d), 1.67-1.22 (12H, m).

### EXAMPLE 22

### 3-Chloro-6-[4-chloro-3-[[(2-cyclohexylethyl)amino]carbonyl]phenyl]-2-pyridinecarboxylic acid

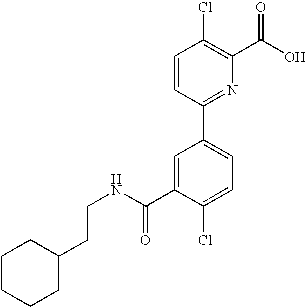

### a) 3-Chloro-6-[4-chloro-3-[(1,1-dimethylethoxy)carbonyl]phenyl]-2-pyridinecarboxylic acid, methyl ester

Prepared according to the method of Example 3 (c) using 2-chloro-5-(4,4,5,5-tetramethyl-1,3,2-dioxaborolan-2-yl)-benzoic acid, 1,1-dimethylethyl ester (Example 3 (b)) (0.8 g) and 3,6-dichloro-2-pyridinecarboxylic acid, methyl ester (0.49 g), stirring at 65° C. under nitrogen for 2 hours. The products were filtered through diatomaceous earth, washing with methanol (2×20 mL) and concentrated in vacuo. The residue was partitioned between dichloromethane (50 mL) and water (25 mL), the layers were separated and the organic fraction was dried (MgSO₄), filtered and concentrated in vacuo. Purification by chromatography (SiO₂, 80:20 isohexane:ethyl acetate as eluant) gave the sub-title compound as a colourless oil (0.82 g).

¹H NMR (400 MHz, CDCl₃) δ 8.31 (1H, d), 8.06 (1H, dd), 7.86 (1H, d), 7.79 (1H, d), 7.52 (1H, d), 4.04 (3H, s), 1.64 (9H, s).

### b) 6-(3-Carboxy-4-chlorophenyl)-3-chloro-2-pyridinecarboxylic acid, 2-methyl ester

Prepared according to the method of Example 3 (d) using 3-Chloro-6-[4-chloro-3-[(1,1-dimethylethoxy)carbonyl]phenyl]-2-pyridinecarboxylic acid, methyl ester (Example 22 (a)) (0.82 g) to give the sub-title compound as an oil (0.69 g).

MS: APCI(+ve) 326/328 (M+H⁺).

### c) 3-Chloro-6-[4-chloro-3-[[(2-cyclohexylethyl)amino]carbonyl]phenyl]-2-pyridinecarboxylic acid, methyl ester

N,N-Dimethylformamide (1 drop) and oxalyl chloride (0.14 mL) were added to a stirred suspension of 6-(3-carboxy-4-chlorophenyl)-3-chloro-2-pyridinecarboxylic acid, 2-methyl ester (Example 22 (b)) (172 mg) in dichloromethane (5 mL). The mixture was stirred under nitrogen for 90 minutes, concentrated in vacuo and dichloromethane (5 mL) was then added, followed by triethylamine (0.22 mL) and cyclohexaneethanamine hydrochloride (129 mg). The mixture was stirred at room temperature for 16 hours, dichloromethane (25 mL) and 2M aqueous hydrochloric acid (10 mL) were added and the layers were separated. The organic fraction was washed with saturated aqueous sodium hydrogen carbonate (10 mL), dried (MgSO₄), filtered and concentrated in vacuo. Purification by chromatography (SiO₂, 99:1 dichloromethane:methanol as eluant) gave the sub-title compound as an oil (160 mg).

MS: APCI(+ve) 435/437 (M+H⁺).

### d) 3-Chloro-6-[4-chloro-3-[[(2-cyclohexylethyl)amino]carbonyl]phenyl]-2-pyridinecarboxylic acid

Prepared according to the method of Example 19 (d) using 3-chloro-6-[4-chloro-3-[[(2-cyclohexylethyl)amino]carbonyl]phenyl]-2-pyridinecarboxylic acid, methyl ester (Example 22 (c)) (160 mg) to give the title compound as a solid (86 mg).

MS: APCI(+ve) 421/423 (M+H⁺).

m.p. 189-190° C.

¹H NMR (300 MHz, d₆-DMSO) δ 13.97 (1H, s), 8.55-8.45 (1H, m), 8.26-8.06 (4H, m), 7.63 (1H, d), 3.30-3.23 (2H, m), 1.80-1.56 (5H, m), 1.49-1.32 (3H, m), 1.29-1.09 (3H, m), 100-0.83 (2H, m).

### EXAMPLE 23

### 3-Chloro-6-[4-chloro-3-[[(cycloheptylmethyl)amino]carbonyl]phenyl]-2-pyridinecarboxylic acid

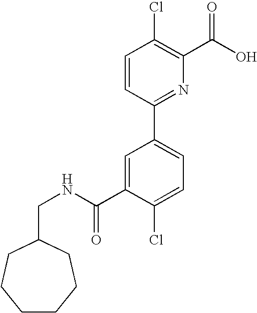

### a) 3-Chloro-6-[4-chloro-3-[[(cycloheptylmethyl)amino]carbonyl]phenyl]-2-pyridinecarboxylic acid, methyl ester

Prepared according to the method of Example 22 (c) using 6-(3-carboxy-4-chlorophenyl)-3-chloro-2-pyridinecarboxylic acid, 2-methyl ester (Example 22 (b)) (172 mg) and cycloheptanemethanamine (100 mg) to give the sub-title compound as a solid (220 mg).

MS: APCI(+ve) 435/437 (M+H⁺).

### b) 3-Chloro-6-[4-chloro-3-[[(cycloheptylmethyl)amino]carbonyl]phenyl]-2-pyridinecarboxylic acid

Prepared according to the method of Example 19 (d) using 3-Chloro-6-[4-chloro-3-[[(cycloheptylmethyl)amino]carbonyl]phenyl]-2-pyridinecarboxylic acid, methyl ester (Example 23 (a)) (220 mg). Further purification by trituration with dichloromethane gave the title compound as a solid (48 mg).

MS: APCI(+ve) 421/423 (M+H⁺).

m.p. 189-190° C.

¹H NMR (400 MHz, d₆-DMSO) δ 13.98 (1H, s), 8.57 (1H, t), 8.24-8.10 (4H, m), 7.63 (1H, d), 3.10 (2H, t), 1.80-1.34 (11H, m), 1.26-1.14 (2H, m).

### EXAMPLE 24

### 3-Chloro-6-[4-chloro-3-[[[(1-hydroxycycloheptyl)methyl]amino]carbonyl]phenyl]-2-pyridinecarboxylic acid

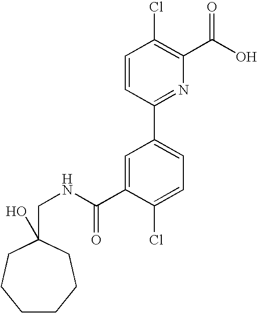

### a) 3-Chloro-6-[4-chloro-3-[[[(1-hydroxycycloheptyl)methyl]amino]carbonyl]phenyl]-2-pyridinecarboxylic acid, methyl ester

Prepared according to the method of Example 22 (c) using 6-(3-carboxy-4-chlorophenyl)-3-chloro-2-pyridinecarboxylic acid, 2-methyl ester (Example 22 (b)) (172 mg) and 1-(aminomethyl)-cycloheptanol (Example 2(b)) (113 mg) to give the sub-title compound as an oil (150 mg).

MS: APCI(−ve) 449 (M−H⁺).

### b) 3-Chloro-6-[4-chloro-3-[[[(1-hydroxycycloheptyl)methyl]amino]carbonyl]phenyl]-2-pyridinecarboxylic acid

Prepared according to the method of Example 19 (d) using 3-chloro-6-[4-chloro-3-[[[(1-hydroxycycloheptyl)methyl]amino]carbonyl]phenyl]-2-pyridinecarboxylic acid, methyl ester (Example 24 (a)) (150 mg). Further purification of the products by RP-HPLC gave the title compound as a solid (46 mg).

MS: APCI(−ve) 435/437 (M−H⁺).

m.p. 146-149° C.

¹H NMR (400 MHz, d₆-DMSO) δ 8.35 (1H, t), 8.23-8.12 (4H, m), 7.63 (1H, d), 3.26 (2H, d), 1.70-1.32 (12H, m).

### EXAMPLE 25

### 1-[3-[4-Chloro-3-[[(2-cyclohexylethyl)amino]carbonyl]phenyl]-2-pyridinyl]-4-piperidinecarboxylic acid

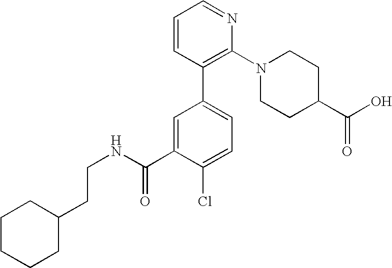

### a) 1-[3-[4-Chloro-3-[(1,1-dimethylethoxy)carbonyl]phenyl]-2-pyridinyl]-4-piperidinecarboxylic acid, methyl ester

Prepared according to the method of Example 3 (c) using 2-chloro-5-(4,4,5,5-tetramethyl-1,3,2-dioxaborolan-2-yl)-benzoic acid, 1,1-dimethylethyl ester (Example 3 (b)) (0.8 g) and 1-(3-bromo-2-pyridinyl)-4-piperidinecarboxylic acid, methyl ester (Example 17 (a)) (0.71 g), stirring at 65° C. under nitrogen for 2 hours. The products were filtered through diatomaceous earth, washing with methanol (2×20 mL) and concentrated in vacuo. The residue was partitioned between dichloromethane (50 mL) and water (25 mL), the layers were separated and the organic fraction was dried (MgSO₄), filtered and concentrated in vacuo. Purification by chromatography (SiO₂, 80:20 isohexane:ethyl acetate as eluant) gave the sub-title compound as a colourless oil (0.82 g).

¹H NMR (400 MHz, CDCl₃) δ 8.24 (1H, dd), 8.01 (1H, d), 7.65 (1H, dd), 7.47 (1H, d), 7.46 (1H, dd), 6.93 (1H, dd), 3.67 (3H, s), 3.52-3.44 (2H, m), 2.72 (2H, dd), 2.42-2.34 (1H, m), 1.88-1.81 (2H, m), 1.69-1.59 (2H, m), 1.62 (9H, s).

### b) 1-[3-(3-Carboxy-4-chlorophenyl)-2-pyridinyl]-4-piperidinecarboxylic acid, 4-methyl ester

Prepared according to the method of Example 3 (d) using 1-[3-[4-Chloro-3-[(1,1-dimethylethoxy)carbonyl]phenyl]-2-pyridinyl]-4-piperidinecarboxylic acid, methyl ester (Example 25 (a)) (0.82 g) to give the sub-title compound as an oil (0.73 g).

MS: APCI(+ve) 375 (M+H⁺).

### c) 1-[3-[4-Chloro-3-[[(2-cyclohexylethyl)amino]carbonyl]phenyl]-2-pyridinyl]-4-piperidinecarboxylic acid, methyl ester

Prepared according to the method of Example 19 (c) using 1-[3-(3-carboxy-4-chlorophenyl)-2-pyridinyl]-4-piperidinecarboxylic acid, 4-methyl ester (Example 25 (b)) (180 mg) and cyclohexaneethanamine hydrochloride (117 mg) to give the sub-title compound as an oil (230 mg).

MS: APCI(+ve) 485/487 (M+H⁺).

### d) 3-Chloro-6-[4-chloro-3-[[(2-cyclohexylethyl)amino]carbonyl]phenyl]-2-pyridinecarboxylic acid

Prepared according to the method of Example 19 (d) using 1-[3-[4-chloro-3-[[(2-cyclohexylethyl)amino]carbonyl]phenyl]-2-pyridinyl]-4-piperidinecarboxylic acid, methyl ester (Example 25 (c)) (230 mg). Purification of the crude products by RP-HPLC gave the title compound as a solid (53 mg).

MS: APCI(−ve) 468 (M−H⁺).

m.p. 116-120° C.

¹H NMR (400 MHz, d₆-DMSO) δ 8.38 (1H, m), 8.20 (1H, dd), 7.72 (1H, dd), 7.61-7.57 (2H, m), 7.55 (1H, d), 7.01 (1H, dd), 3.38 (2H, d), 3.26 (2H, td), 2.67 (2H, t), 2.28 (1H, t), 1.78-0.83 (17H, m).

### EXAMPLE 26

### 1-[3-[4-Chloro-3-[[(cycloheptylmethyl)amino]carbonyl]phenyl]-2-pyridinyl]-4-piperidinecarboxylic acid

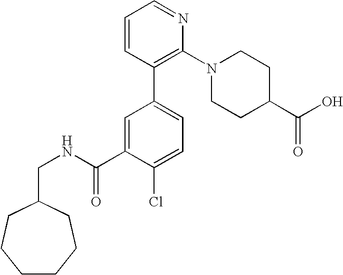

### a) 1-[3-[4-Chloro-3-[[(cycloheptylmethyl)amino]carbonyl]phenyl]-2-pyridinyl]-4-piperidinecarboxylic acid, methyl ester

Prepared according to the method of Example 19 (c) using 1-[3-(3-carboxy-4-chlorophenyl)-2-pyridinyl]-4-piperidinecarboxylic acid, 4-methyl ester (Example 25 (b)) (180 mg) and cycloheptanemethanamine (91 mg) to give the sub-title compound as an oil (210 mg).

MS: APCI(+ve) 485/487 (M+H⁺).

### b) 1-[3-[4-Chloro-3-[[(cycloheptylmethyl)amino]carbonyl]phenyl]-2-pyridinyl]-4-piperidinecarboxylic acid

Prepared according to the method of Example 19 (d) using 1-[3-[4-chloro-3-[[(cycloheptylmethyl)amino]carbonyl]phenyl]-2-pyridinyl]-4-piperidinecarboxylic acid, methyl ester (Example 26 (a)) (210 mg). Purification of the crude products by chromatography (SiO₂, 96:4 dichloromethane:methanol as eluant) and then by Varian NH₂ cartridge using methanol (100 mL) and then 2% trifluoroacetic acid in methanol (100 mL) as eluant gave the title compound as a solid (42 mg).

MS: APCI(+ve) 470 (M+H⁺).

m.p. 164-167° C.

¹H NMR (400 MHz, d₆-DMSO) δ 8.45 (1H, t), 8.21 (1H, dd), 7.72-7.66 (2H, m), 7.62 (1H, d), 7.57 (1H, d), 7.11-7.05 (1H, m), 3.40 (2H, d), 3.07 (2H, t), 2.74 (2H, t), 2.38-2.29 (1H, m), 1.79-1.13 (17H, m).

### EXAMPLE 27

### 1-[3-[4-Chloro-3-[[[(1-hydroxycycloheptyl)methyl]amino]carbonyl]phenyl]-2-pyridinyl]-4-piperidinecarboxylic acid

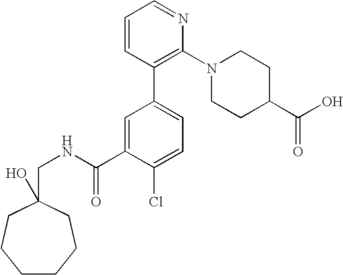

### a) 1-[3-[4-Chloro-3-[[[(1-hydroxycycloheptyl)methyl]amino]carbonyl]phenyl]-2-pyridinyl]-4-piperidinecarboxylic acid, methyl ester

Prepared according to the method of Example 19 (c) using 1-[3-(3-carboxy-4-chlorophenyl)-2-pyridinyl]-4-piperidinecarboxylic acid, 4-methyl ester (Example 25 (b)) (180 mg) and 1-(aminomethyl)-cycloheptanol (Example 2 (b)) (69 mg) to give the sub-title compound as an oil (220 mg).

MS: APCI(−ve) 499/501 (M−H⁺).

### b) 1-[3-[4-Chloro-3-[[[(1-hydroxycycloheptyl)methyl]amino]carbonyl]phenyl]-2-pyridinyl]-4-piperidinecarboxylic acid

Prepared according to the method of Example 19 (d) using 1-[3-[4-chloro-3-[[[(1-hydroxycycloheptyl)methyl]amino]carbonyl]phenyl]-2-pyridinyl]-4-piperidinecarboxylic acid, methyl ester (Example 27 (a)) (220 mg). Purification of the crude products by chromatography (SiO₂, 92:8 dichloromethane:methanol as eluant) and then by RP-HPLC gave the title compound as a solid (21 mg).

MS: APCI(−ve) 484 (M−H⁺).

m.p. 117-119° C.

¹H NMR (400 MHz, d₆-DMSO) δ 8.24 (1H, t), 8.21 (1H, dd), 7.72 (1H, dd), 7.67 (1H, d), 7.61 (1H, dd), 7.55 (1H, d), 7.02 (1H, dd), 4.26 (1H, s), 3.43-3.35 (2H, m), 3.23 (2H, d), 2.67 (2H, t), 2.35-2.25 (1H, m), 1.77-1.22 (16H, m).

### Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantif the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing concentrations of test compound typically from 30 μM-0.001 μM. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure >5.5. For example, the following table shows the pIC₅₀ figures for a representative selection of compounds:

| Compound of | |
|---|---|
| Example No. | pIC₅₀ |
| 2 | 7.2 |
| 26 | 7.0 |

1. A compound of general formula (I), or a pharmaceutically acceptable salt thereof, 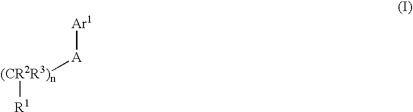
wherein Ar¹ represents a group 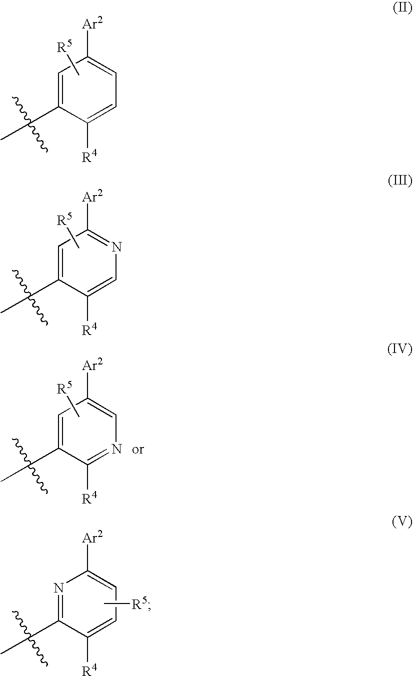
A represents C(O)NH or NHC(O);
R¹ represents a 3- to 9-membered carbocyclic or 4- to 10-membered heterocyclic ring, which carbocyclic ring or heterocyclic ring can be optionally substituted by at least one substituent independently selected from halogen, cyano, nitro, NR⁶R⁷, C₁₋₆ alkylsulphonyl, C₁₋₆ alkoxy and C₁₋₆ alkyl group which C₁₋₆ alkyl group can be optionally substituted by at least one substituent independently selected from halogen and hydroxyl;
n is 0, 1, 2 or 3;
within each grouping, CR²R³, R² and R³ each independently represents hydrogen, halogen, phenyl or a C₁₋₆ alkyl group, or R² and R³ together with the carbon atom to which they are both attached form a 3- to 8-membered cycloalkyl ring;
one of R⁴ and R⁵ represents halogen, nitro, NR⁶R⁷, hydroxyl, C₁₋₆ alkoxy optionally substituted by at least one halogen, or a C₁₋₆ alkyl group optionally substituted by at least one halogen, and the other of R⁴ and R⁵ represents hydrogen, halogen or a C₁₋₆ alkyl group optionally substituted by at least one halogen;
Ar² represents phenyl substituted by at least one substituent independently selected from carboxyl, MC₁₋₆ alkylCO₂H, C₁₋₆ alkylsulphonylaminocarbonyl, C(O)NHOH, NHR⁸, R⁹, XR¹⁰ and NR¹⁷R¹⁸, or Ar² represents a 5- or 6-membered heteroaromatic ring comprising from 1 to 2 heteroatoms independently selected from nitrogen, oxygen and sulphur, which heteroaromatic ring is substituted by at least one substituent independently selected from carboxyl, MC₁₋₆ alkylCO₂H, C₁₋₆ alkylsulphonylaminocarbonyl, C(O)NHOH, NHR⁸ and NR¹⁹R²⁰;
wherein the phenyl or heteroaromatic ring Ar² can further be optionally substituted by at least one substituent independently selected from halogen, nitro, NR⁶R⁷, S(O)₀₋₂R¹¹, C₁₋₆ alkoxy one substituted independently selected from halogen, nitro, NR⁶R⁷, S(O)₀₋₂R¹¹, C₁₋₆ alkoxy optionally substituted by at least one halogen, and a C₁₋₆ alkyl group which C₁₋₆ alkyl group can be optionally substituted by at least one substituted independently selected from halogen, hydroxyl, NR⁶R⁷, SO₂NR⁶R⁷, NR¹¹SO₂R¹¹, NHCOR¹¹ and CONR⁶R⁷;
R⁸ represents CN, C₁₋₆ alkoxycarbonyl, C₁₋₆ alkylaminosulphonyl, or (di)-C₁₋₆ alkylaminosulphonyl;
R⁹ and R¹⁰ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen and sulphur, which heterocyclic ring is substituted by at least one substituent independently selected from hydroxyl, ═O and ═S, and which heterocyclic ring may further be optionally substituted by at least one substituent independently selected from halogen, nitro amino, cyano, C₁₋₆ alkylsulphonyl, C₁₋₆alkoxycarbonyl and C₁₋₆ alkyl group which C₁₋₆ alkyl group can be optionally substituted by at least one substituted independently selected from halogen, hydroxyl and amino;
M represents a bond, oxygen, S(O)₀₋₂ or NR¹¹;
X represents oxygen, S(O)₀₋₂ or NR¹¹, C₁₋₆ alkylene, O(CH₂)₁₋₆, NR¹¹(CH₂)₁₋₆ or S(O)₀₋₂ (CH₂)₁₋₆;
R⁶ and R⁷ each independently represent a hydrogen atom or a C₁₋₆ alkyl group optionally substituted by at least one substituent independently selected from hydroxyl, halogen and C₁₋₆ alkoxy, or R⁶ and R⁷ together with the nitrogen atom to which they are attached form a 3- to 8-membered saturated heterocyclic ring;
R¹¹ represents a hydrogen atom or a C₁₋₆ alkyl group optionally substituted by at least one substituent independently selected from hydroxyl, halogen and C₁₋₆ alkoxy;
R¹⁷ and R¹⁸ together with the nitrogen atom to which they are attached form a 3- to 8-membered saturated heterocyclic ring; which heterocyclic ring is substituted with at least one substituent independently selected from carboxyl, MC₁₋₆ alkylCO₂H, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituted independently selected from hydroxyl, halogen, C₁₋₆ alkoxy optionally substituted by at least one halogen, and C₁₋₆ alkyl group which C₁₋₆ alkyl group can be optionally substituted by at least one substituted independently selected from halogen and hydroxyl;
R¹⁹ and R²⁰ together with the nitrogen atom to which they are attached form a 3- to 8-membered saturated heterocyclic ring, which heterocyclic ring is substituted with at least one substituent independently selected from carboxyl, MC₁₋₆ alkylCO₂H, C₁₋₆ alkylsulphonylaminocarbonyl, C(O)NHOH and NHR⁸, and which 3- to 8-membered saturated heterocyclic ring can further be optionally substituted by at least one substituted independently selected from hydroxyl, halogen, C₁₋₆ alkyl optionally substituted by at least one halogen, and C₁₋₆ alkyl group which C₁₋₆ alkyl group can be optionally substituted by at least one substituted independently selected from halogen and hydroxyl;
provided that the compound of formula (I) is not
(3-{4-chloro-3-[(1-hydroxy-cycloheptylmethyl)-carbamoyl]-phenyl}-5-methyl-pyrazol-1-yl)-acetic acid
(3-[4-methoxy-3-({[4-(trifluoromethyl)benzyl]amino}carbamoyl)-phenyl]benzoic acid, or
(3-[4-methoxy-3-({[2,4-dichlorobenzyl]amino}carbonyl)phenyl]benzoic acid.

2. A compound according to claim 1, wherein A represents NHC(O).

3. A compound according to claim 1, wherein Ar² represents phenyl or pyridyl.

4. A compound according to claim 1, wherein Ar² is substituted by a substituent selected form carboxyl, MC₁₋₆ alkylCO₂H and C₁₋₆ alkylsulphonylaminocarbonyl.

5. A compound according to claim 1, wherein Ar¹ represents a group.

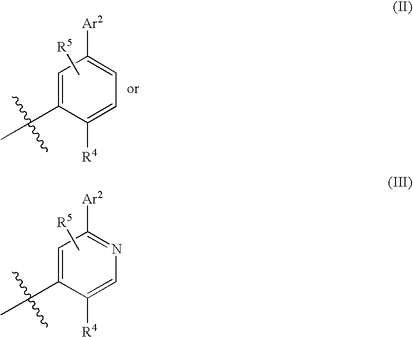

6. A compound according to claim 1, wherein R⁴ represents halogen, nitro, NH₂, hydroxyl, or a C₁₋₄ alkyl optionally substituted by one to three halogen substituents, and R⁵ represents a hydrogen atom.

7. A compound according to claim 1 of general formula (I), or a pharmaceutically acceptable salt thereof, 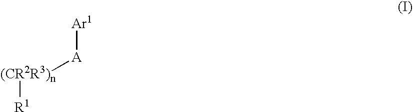
wherein Ar¹ represents a group 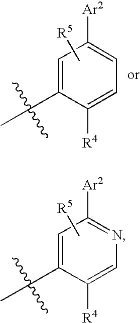
A represents NHC(O);
R¹ represents phenyl or a 3- to 9-membered aliphatic carbocyclic ring, which phenyl or aliphatic carbocyclic ring can be optionally substituted by at least one substituent independently selected from halogen, hydroxyl and a C₁₋₄ alkyl group which C₁₋₄ alkyl group can be optionally substituted by hydroxyl;
n is 0, 1 or 2;
within each grouping, CR²R³, R² and R³ each independently represent hydrogen, or a C₁₋₄alkyl group;
one of R⁴ and R⁵ represents halogen, nitro, NR⁶R⁷, hydroxyl, or a C₁₋₆alkyl group optionally substituted by at least one halogen, and the other of R⁴ and R⁵ represents hydrogen;
Ar² represents phenyl substituted by at least one substituent independently selected from carboxyl and NR¹⁷R¹⁸,
or Ar² represents pyridyl substituted by at least one substituent independently selected from carboxyl and NR¹⁹R²⁰,
R⁶ and R⁷ each independently represent a hydrogen atom or a C₁₋₆ alkyl group;
R¹⁷ and R¹⁸ together with the nitrogen atom to which they are attached form a 6-membered saturated heterocyclic ring, which heterocyclic ring is substituted with at least one substituent independently selected from carboxyl and C₁₋₆ alkylCO₂H; and
R¹⁹ and R²⁰ together with the nitrogen atom to which they are attached form a 6-membered saturated heterocyclic ring, which heterocyclic ring is substituted with at least one substituent independently selected from carboxyl and C₁₋₆ alkylCO₂H.

8. A compound according to claim 1, which is selected from
4′-Chloro-3′-[[[2-(2-chlorophenyl)ethyl]amino]carbonyl]-[1,1′-biphenyl]-2-carboxylic acid,
4′-Chloro-3′-[[[(1-hydroxycycloheptyl)methyl]amino]carbonyl]-[1,1′-biphenyl]-2-carboxylic acid,
4′-Chloro-3′-[[(cyclohexylmethyl)amino]carbonyl]-[1,1′-biphenyl]-2-carboxylic acid,
4′-Chloro-3′-[[[(2S)-2-phenylpropyl]amino]carbonyl]-[1,1′-biphenyl]-2-carboxylic acid,
4′-Chloro-3′-[[[[(1S,2R,5S)-6,6-dimethylbicyclo[3.1.1]hept-2-yl]methyl]amino]carbonyl]-[1,1′-biphenyl]-2-carboxylic acid,
4′-Chloro-3′-[[(cycloheptylmethyl)amino]carbonyl]-[1,1′-biphenyl]-2-carboxylic acid,
4′-Chloro-3′-[[[(1-hydroxycyclohexyl)methyl]amino]carbonyl]-[1,1′-biphenyl]-2-carboxylic acid,
4′-Chloro-3′-[[[[cis-2-hydroxycycloheptyl]methyl]amino]carbonyl]-[1,1′-biphenyl]-2-carboxylic acid,
4′-Chloro-3′-[[(2-cyclohexylethyl)amino]carbonyl]-[1,1′-biphenyl]-2-carboxylic acid,
3-[4-Chloro-3-[[(cycloheptylmethyl)amino]carbonyl]phenyl]-2-pyridinecarboxylic acid,
3-[4-Chloro-3-[[(2-cyclohexylethyl)amino]carbonyl]phenyl]-2-pyridinecarboxylic acid
4′-Chloro-3′-[[[(1R)-1-cyclohexylethyl]amino]carbonyl]-[1,1′-biphenyl]-2-carboxylic acid,
4′-Chloro-3′-[[[(1-methylcycloheptyl)methyl]amino]carbonyl]-[1,1′-biphenyl]-2-carboxylic acid,
4′-Chloro-3′-[[[[1-hydroxymethyl)cycloheptyl]methyl]amino]carbonyl]-[1,1′-biphenyl]-2-carboxylic acid,
3-[4-Chloro-3-[[[(1-hydroxycycloheptyl)methyl]amino]carbonyl]phenyl]-2-pyridinecarboxylic acid,
3′-[[(Cycloheptylmethyl)amino]carbonyl]-4′-methyl-[1,1′-biphenyl]-2-carboxylic acid,
1-[3-[3-[[(Cycloheptylmethyl)amino]carbonyl]-4-methylphenyl]-2-pyridinyl]-4-piperidinecarboxylic acid,
3-Chloro-6-[3-[[(cycloheptylmethyl)amino]carbonyl]-4-methylphenyl]-2-pyridinecarboxylic acid,
5-Chloro-2-[4-chloro-3-[[(2-cyclohexylethyl)amino]carbonyl]phenyl]-3-pyridinecarboxylic acid,
5-Chloro-2-[4-chloro-3-[[(cycloheptylmethyl)amino]carbonyl]phenyl]-3-pyridinecarboxylic acid,
5-Chloro-2-[4-chloro-3-[[[(1-hydroxycycloheptyl)methyl]amino]carbonyl]phenyl]-3-pyridinecarboxylic acid,
3-Chloro-6-[4-chloro-3-[[(2-cyclohexylethyl)amino]carbonyl]phenyl]-2-pyridinecarboxylic acid,
3-Chloro-6-[4-chloro-3-[[(2-cycloheptylmethyl)amino]carbonyl]phenyl]-2-pyridinecarboxylic acid,
3-Chloro-6-[4-chloro-3-[[[(1-hydroxycycloheptyl)methyl]amino]carbonyl]phenyl]-2-pyridinecarboxylic acid,
1-[3-[4-Chloro-3-[[(2-cyclohexylethyl)amino]carbonyl]phenyl]-2-pyridinyl]-4-piperidinecarboxylic acid,
1-[3-[4-Chloro-3-[[(cycloheptylmethyl)amino]carbonyl]phenyl]-2-pyridinyl]-4-piperidinecarboxylic acid,
1-[3-[4-Chloro-3-[[[(1-hydroxycycloheptyl)methyl]amino]carbonyl]phenyl]-2-pyridinyl]-4-piperidinecarboxylic acid,
or a pharmaceutically acceptable salt thereof.

9. A process for the preparation of a compound of formula (I) as defined in claim 1, or a pharmaceutically acceptable salt thereof, which comprises:
(a) reacting a compound of formula 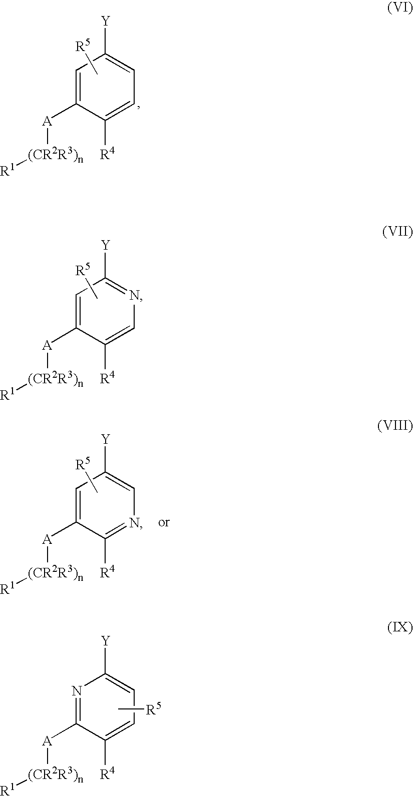
with a compound of formula
Z-Ar² (X)
wherein one of Y and Z represents a displaceable group such as a metallic, organometallic or organosilicon group and the other of Y and Z represent a leaving group such as a halogeno or sulphonyloxy group and Ar², R¹, R², R³, n, A, R⁴ and R⁵ are as defined in formula (I); or
(b) when Ar² is substituted by carboxyl, reacting a compound of formula (VI)-(IX) as defined in (a) above with a compound of formula
Z-Ar^{2a}—CO₂R¹² (XI)
wherein Z is as defined in formula (X), Ar^{2a} represents a phenyl or 5- or 6-membered heteroaromatic ring comprising from 1 to 2 heteroatoms independently selected from nitrogen, oxygen and sulphur, and R¹² is a C₁₋₆ alkyl group, followed by reaction with a base; or
(c) when Ar² is substituted by carboxyl, reacting a compound of formula (VI)-(IX) as defined in (a) above with a compound of formula
Z-Ar^{2b}—CN (XII)
wherein Z is as defined in formula (X), Ar^{2a} represents a phenyl or 5- or 6-membered heteroaromatic ring comprising from 1 to 2 heteroatoms independently selected from nitrogen, oxygen and sulphur, followed by reaction with a base; or
(d) when R⁸ represents CN, C₁₋₆alkoxycarbonyl, C₁₋₆alkylaminosulphonyl, or (di)-C₁₋₆ alkylaminosulphonyl, reacting a compound of formula (VI)-(IX) as defined in (a) above with a compound of formula
L¹-Ar^{2c}-Z (XIII)
wherein L¹ represents a leaving group such as a halogen or sulphonyloxy group, Ar^{2c} represents a phenyl or 5- or 6-membered heteroaromatic ring comprising from 1 to 2 heteroatoms independently selected from nitrogen, oxygen and sulphur, and Z is as defined in formula (X), followed by reaction with a compound of formula 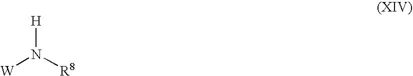
wherein W represents a hydrogen or a metallic group and R⁸ is as defined in formula (I); or
(e) when Ar² is substituted by carboxyl, reacting a compound of formula (VI)-(IX) as defined in (a) above with a compound of formula (XIII) as defined in (d) above, followed by reaction with a suitable source of cyanide, followed by reacting with a base; or
(f) when Ar² is substituted by carboxyl, reacting a compound of formula (VI)-(IX) as defined in (a) above with a compound of formula (XIII) as defined in (d) above followed by reaction with carbon monoxide and an alcohol in the presence of a suitable catalyst, for example a palladium catalyst, followed by reaction with a base; or
(g) reacting a compound of formula 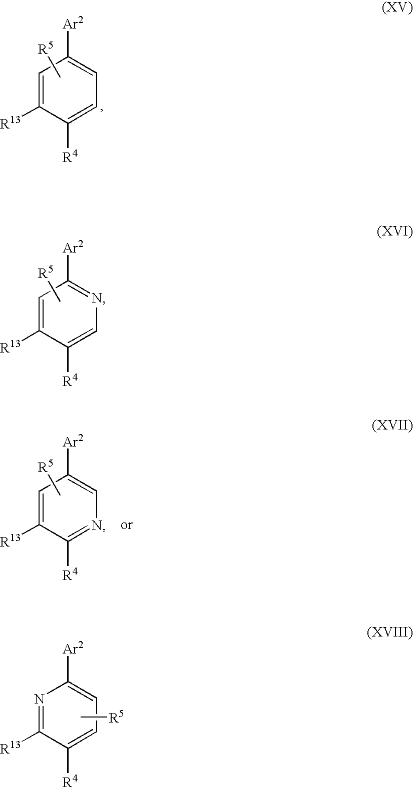
with a compound of formula 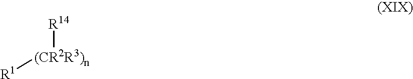
wherein one of R¹³ and R¹⁴ represents NH₂ and the other of R¹³ and R¹⁴ represents CO₂H, COBr or COCl, and R¹, R², R³, n, R⁴, R⁵ and Ar² are as defined in formula (I); or
(h) reacting a compound of formula 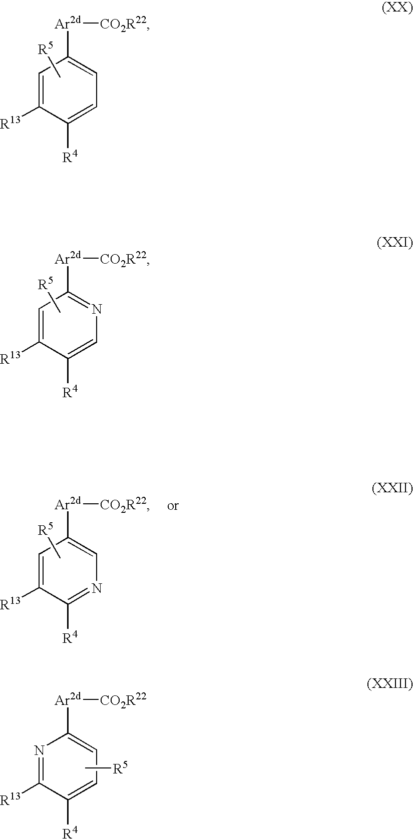
with a compound of formula (XIX) as defined in (g) above, wherein Ar^{2d} represents a phenyl or 5- or 6-membered heteroaromatic ring comprising from 1 to 2 heteroatoms independently selected from nitrogen, oxygen and sulphur, R¹² is as defined in formula (XI), R⁴ and R⁵ are as defined in formula (I), and R¹³ is as defined in formula (XV)-(XVIII), followed by reaction with a base, or acid; or
(i) when R¹⁹ and R²⁰ together with the nitrogen to which they are attached form a 3- to 8-membered saturated heterocyclic ring, which heterocyclic ring is substituted by carboxyl, reacting a compound of formula (VI)-(IX) as defined in (a) above wherein Y represents a displaceable group such as an organoboron group, with a compound of formula 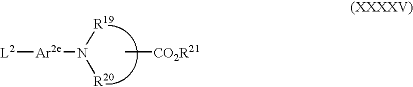
wherein R²¹ represents a C₁₋₆alkyl group, Ar^{2c} represents a 5- or 6-membered heteroaromatic ring comprising from 1 to 2 heteroatoms independently selected from nitrogen, oxygen and sulphur, L² represents a leaving group such as a halogeno or sulphonyloxy group and R¹⁹ and R²⁰ are as defined in formula (I), optionally followed by reaction with a base or an acid; or
(j) when R¹⁹ and R²⁰ together with the nitrogen to which they are attached form a 3- to 8-membered saturated heterocyclic ring, which heterocylic ring is substituted by carboxyl, reacting a compound of formula (XIX) as defined in (g) above, with a compound of formula 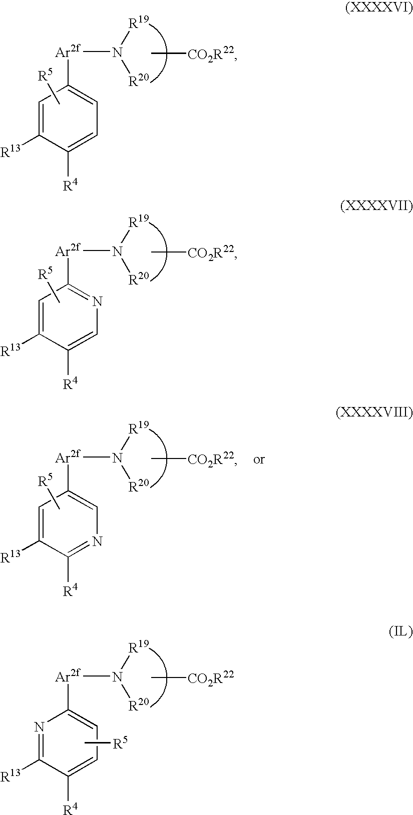
wherein Ar^{2f} represents a 5- or 6-membered heteroaromatic ring comprising from 1 to 2 heteroatoms independently selected from nitrogen, oxygen and sulphur, R²² is a C₁₋₆alkyl group R⁴, R⁵, R¹⁹ and R²⁰ are as defined in formula (I), and R¹³ is as defined in formula (XV)-(XVIII), formula by reaction with a base or an acid;
and optionally after (a), (b), (c), (d), (e), (f), (g), (h), (i), or (j) carrying out one or more of the following:
converting the compound to a further compound of the invention
forming a pharmaceutically acceptable salt of the compound.

10. A process composition comprising a compound of formula (I), or a pharmaceutically acceptable salt thereof, as claimed in claim 1 in association with a pharmaceutically acceptable adjuvant, diluent or carrier.

11. A process for the preparation of a pharmaceutical composition comprising a compound of formula (I), or a pharmaceutically acceptable salt thereof in association with a pharmaceutically acceptable adjuvant, diluent or carrier, which comprises mixing a compound of formula (I), or a pharmaceutically acceptable salt thereof, as defined in claim 1 with a pharmaceutically acceptable adjuvant, diluent or carrier.

12. (canceled)

13. A method of treating rheumatoid arthritis, the method comprising administering to a subject a compound of formula (I), or a pharmaceutically acceptable salt thereof, as claimed in claim 1.

14. A method of treating osteoarthritis, the method comprising administering to a subject a compound of formula (I), or a pharmaceutically acceptable salt thereof, as claimed in claim 1.

15. A method of treating asthma or chronic obstructive pulmonary disease, the method comprising administering to a subject a compound of formula (I), or a pharmaceutically acceptable salt thereof, as claimed in claim 1.

16. A method of treating atherosclerosis, the method comprising administering to a subject a compound of formula (I), or a pharmaceutically acceptable salt thereof, as claimed in claim 1.
